(19) United States
(12) Patent Application Publication
(10) Pub. No.: US 2023/0285519 A1
(43) Pub. Date: Sep. 14, 2023

(54) B-GLUCURONIDASE PROTEINS HAVING PNEUMOCOCCAL CAPSULE DEGRADING ACTIVITY AND METHODS OF USE

(71) Applicant: UNIVERSITY OF GEORGIA RESEARCH FOUNDATION, INC., Athens, GA (US)

(72) Inventors: Fikri Y. Avci, Watkinsville, GA (US); Paeton Wantuch, Athens, GA (US)

(21) Appl. No.: 18/016,511

(22) PCT Filed: Jul. 16, 2021
(86) PCT No.: PCT/US2021/041932

§ 371 Date: Jan. 17, 2023

Related U.S. Application Data
(60) Provisional application No. 63/053,166, filed on Jul. 17, 2020.

(51) Int. Cl. A61K 38/47; C12N 9/24; A61P 31/04

(52) U.S. Cl. CPC A61K 38/47; A61P 31/04; C12N 9/2402; C12Y 302/01031

(57) ABSTRACT

The present disclosure includes catalytically active truncations of a protein, referred to as a PnSPase protein, that degrade the capsular polysaccharide of serotype 3 Streptococcus pneumoniae. The disclosure includes a genetically modified cell that includes a PnSPase protein of the present disclosure, and compositions that include the protein, the polynucleotide encoding the protein, the genetically modified cell, or a combination thereof. Also provided are methods for using a PnSPase protein of the present disclosure, including methods for contacting a S. pneumoniae having a type III capsular polysaccharide with a PnSPase protein, increasing deposition of at least one complement component on the surface of a S. pneumoniae, treating an infection in a subject, treating a symptom in a subject, decreasing colonization of a subject by S. pneumoniae, or a combination thereof.

### CROSS-REFERENCE TO RELATED APPLICATIONS

This application claims the benefit of U.S. Provisional Application Serial No. 63/053,166, filed Jul. 17, 2020, which is incorporated by reference herein in its entirety.

### SEQUENCE LISTING

This application contains a Sequence Listing electronically submitted via EFS-Web to the United States Patent and Trademark Office as an ASCII text file entitled “Seq_List_0235-000293WOO1_ST25” having a size of 54 kilobytes and created on Jul. 13, 2021. The information contained in the Sequence Listing is incorporated by reference herein.

### GOVERNMENT FUNDING

This invention was made with government support under R01AI123383 awarded by the National Institutes of Health. The government has certain rights in the invention.

### SUMMARY OF THE APPLICATION

This disclosure describes catalytically active truncations of a protein, referred to as a Pn3Pase protein, that degrade the capsular polysaccharide of serotype 3 Streptococcus pneumoniae. The disclosure includes a genetically modified cell that includes a Pn3Pase protein, and compositions that include the protein, the polynucleotide encoding the protein, the genetically modified cell, or a combination thereof. Also provided are methods for using a Pn3Pase protein of the present disclosure, including methods for contacting a S. pneumoniae having a type III capsular polysaccharide with a Pn3Pase protein, increasing deposition of at least one complement component on the surface of a S. pneumoniae, treating an infection in a subject, treating a symptom in a subject, decreasing colonization of a subject by S. pneumoniae, or a combination thereof.

Terms used herein will be understood to take on their ordinary meaning in the relevant art unless specified otherwise. Several terms used herein and their meanings are set forth below.

As used herein, “genetically modified cell” refers to a cell into which has been introduced an exogenous polynucleotide, such as an expression vector. For example, a cell is a genetically modified cell by virtue of introduction into a suitable cell of an exogenous polynucleotide that is foreign to the cell. “Genetically modified cell” also refers to a cell that has been genetically manipulated such that endogenous nucleotides have been altered. For example, a cell is a genetically modified cell by virtue of introduction into a suitable cell of an alteration of endogenous nucleotides. An example of a genetically modified cell is one having an altered regulatory sequence, such as a promoter, to result in increased or decreased expression of an operably linked endogenous coding region.

As used herein, the term “protein” refers broadly to a polymer of two or more amino acids joined together by peptide bonds. The term “protein” also includes molecules which contain more than one protein joined by a disulfide bond, or complexes of proteins that are joined together, covalently or noncovalently, as multimers (e.g., dimers, tetramers). Thus, the terms peptide, oligopeptide, enzyme, and polypeptide are all included within the definition of protein and these terms are used interchangeably. It should be understood that these terms do not connote a specific length of a polymer of amino acids, nor are they intended to imply or distinguish whether the protein is produced using recombinant techniques, chemical or enzymatic synthesis, or is naturally occurring.

As used herein, an “isolated” substance, for instance a protein, is one that has been removed from its natural environment, produced using recombinant techniques, or chemically or enzymatically synthesized. A protein produced by a genetically modified cell is not considered isolated. For instance, a protein or a polynucleotide can be isolated. Preferably, a substance is purified, i.e., is at least 60% free, preferably at least 75% free, and most preferably at least 90% free from other components with which they are naturally associated.

As used herein, the term “polynucleotide” refers to a polymeric form of nucleotides of any length, either ribonucleotides or deoxynucleotides, and includes both double- and single-stranded RNA and DNA. A polynucleotide can be obtained directly from a natural source, or can be prepared with the aid of recombinant, enzymatic, or chemical techniques. A polynucleotide can be linear or circular in topology. A polynucleotide may be, for example, a portion of a vector, such as an expression or cloning vector, or a fragment. A polynucleotide may include nucleotide sequences having different functions, including, for instance, coding regions, and non-coding regions such as regulatory regions.

As used herein, a “detectable moiety” or “label” is a molecule that is detectable, either directly or indirectly, by spectroscopic, photochemical, biochemical, immunochemical, or chemical means. For example, useful labels include ³²P, fluorescent dyes, electron-dense reagents, enzymes and their substrates (e.g., as commonly used in enzyme-linked immunoassays, e.g., alkaline phosphatase and horse radish peroxidase), biotin-streptavidin, digoxigenin, proteins such as antibodies, or haptens and proteins for which antisera or monoclonal antibodies are available. The label or detectable moiety is typically bound, either covalently, through a linker or chemical bound, or through ionic, van der Waals or hydrogen bonds to the molecule to be detected.

As used herein, the terms “coding region” and “coding sequence” are used interchangeably and refer to a nucleotide sequence that encodes a protein and, when placed under the control of appropriate regulatory sequences expresses the encoded protein. The boundaries of a coding region are generally determined by a translation start codon at its 5′ end and a translation stop codon at its 3′ end. A “regulatory sequence” is a nucleotide sequence that regulates expression of a coding sequence to which it is operably linked. Non-limiting examples of regulatory sequences include promoters, enhancers, transcription initiation sites, translation start sites, translation stop sites, and transcription terminators. The term “operably linked” refers to a juxtaposition of components such that they are in a relationship permitting them to function in their intended manner. A regulatory sequence is “operably linked” to a coding region when it is joined in such a way that expression of the coding region is achieved under conditions compatible with the regulatory sequence.

A polynucleotide that includes a coding region may include heterologous nucleotides that flank one or both sides of the coding region. As used herein, “heterologous nucleotides” refer to nucleotides that are not normally present flanking a coding region that is present in a wild-type cell. Thus, a polynucleotide that includes a coding region and heterologous nucleotides is not a naturally occurring molecule. For instance, in those embodiments where a coding region encodes a truncated Pn3Pase protein, any nucleotide sequence other than the nucleotides normally present in a wild-type cell flanking the coding region of the truncated Pn3Pase protein are considered to be heterologous. Examples of heterologous nucleotides include, but are not limited to, regulatory sequences. Typically, heterologous nucleotides are present in a polynucleotide described herein through the use of standard genetic and/or recombinant methodologies well known to one skilled in the art. A polynucleotide described herein may be included in a suitable vector.

A protein described herein may include heterologous amino acids present at the N-terminus, the C-terminus, or a combination thereof. As used herein, “heterologous amino acids” refer to amino acids that are not normally present flanking a protein that is naturally present in a wild-type cell. Thus, a protein that includes heterologous amino acids is not a naturally occurring molecule. For instance, for a Pn3Pase protein of the present disclosure any amino acid sequence other than the amino acids normally flanking the truncated protein (e.g., in one embodiment described in detail herein, amino acids 41-765 of SEQ ID NO:2) are considered heterologous. Examples of heterologous amino acid sequences are described herein, and include, but are not limited to affinity purification tags. Typically, heterologous amino acids are present in a protein described herein through the use of standard genetic and/or recombinant methodologies well known to one skilled in the art.

A protein described herein may include homologous amino acids present at the N-terminus, the C-terminus, or a combination thereof. As used herein, “homologous amino acids” refer to amino acids that are normally present flanking a protein that is naturally present in a wild-type cell. For instance, for a Pn3Pase protein of the present disclosure, any amino acid sequence that normally flanks the truncated protein (e.g., in one embodiment described in detail herein, amino acids 41-765 of SEQ ID NO:2) is considered homologous. In one embodiment, examples of homologous amino acid sequences include, but are not limited, amino acids 1-40 of SEQ ID NO:2 at the amino terminal end of a Pn3Pase protein of the present disclosure (e.g., amino acids 41-765 of SEQ ID NO:2). Accordingly, a Pn3Pase protein of the present disclosure can include one homologous amino acid at the N-terminal end (amino acid 40 of SEQ ID NO:2), two homologous amino acids at the N-terminal end (amino acids 39-40 of SEQ ID NO:2), three homologous amino acids at the N-terminal end (amino acids 38-40 of SEQ ID NO:2), and so on up to all of amino acids 1-40 of SEQ ID NO:2. In another embodiment, examples of homologous amino acid sequences include, but are not limited to, amino acids 766-1545 of SEQ ID NO:2 at the carboxy terminal end of a Pn3Pase protein of the present disclosure (e.g., amino acids 766-1545 of SEQ ID NO:2). Accordingly, a Pn3Pase protein of the present disclosure can include one homologous amino acid at the C-terminal end (amino acid 766 of SEQ ID NO:2), two homologous amino acids at the C-terminal end (amino acids 766-767 of SEQ ID NO:2), three homologous amino acids at the C-terminal end (amino acids 766-769 of SEQ ID NO:2), and so on up to all of amino acids 766-1545 of SEQ ID NO:2. A Pn3Pase protein of the present disclosure does not include the full length protein, for instance, SEQ ID NO:2 or amino acids 41-1545 of SEQ ID NO:2. Because Pn3Pase protein of the present disclosure does not include the full length protein, the proteins described herein are also referred to as non-natural proteins.

Typically, heterologous amino acids are present in a protein described herein through the use of standard genetic and/or recombinant methodologies well known to one skilled in the art.

As used herein, “exogenous polynucleotide” and “exogenous protein” refers to a polynucleotide or protein, respectively, that is not normally or naturally found in a cell.

The terms “complement” and “complementary” as used herein, refer to the ability of two single stranded polynucleotides to base pair with each other, where an adenine on one strand of a polynucleotide will base pair to a thymine or uracil on a strand of a second polynucleotide and a cytosine on one strand of a polynucleotide will base pair to a guanine on a strand of a second polynucleotide. Two polynucleotides are complementary to each other when a nucleotide sequence in one polynucleotide can base pair with a nucleotide sequence in a second polynucleotide. For instance, 5′-ATGC and 5′-GCAT are complementary. The term “substantial complement” and cognates thereof as used herein refer to a polynucleotide that is capable of selectively hybridizing to a specified polynucleotide under stringent hybridization conditions. Stringent hybridization can take place under a number of pH, salt, and temperature conditions. The pH can vary from 6 to 9, preferably 6.8 to 8.5. The salt concentration can vary from 0.15 M sodium to 0.9 M sodium, and other cations can be used as long as the ionic strength is equivalent to that specified for sodium. The temperature of the hybridization reaction can vary from 30° C. to 80° C., preferably from 45° C. to 70° C. Additionally, other compounds can be added to a hybridization reaction to promote specific hybridization at lower temperatures, such as at or approaching room temperature. Among the compounds contemplated for lowering the temperature requirements is formamide. Thus, a polynucleotide is typically substantially complementary to a second polynucleotide if hybridization occurs between the polynucleotide and the second polynucleotide. As used herein, “specific hybridization” refers to hybridization between two polynucleotides under stringent hybridization conditions.

In the comparison of two amino acid sequences, structural similarity may be referred to by percent “identity” or may be referred to by percent “similarity.” “Identity” refers to the presence of identical amino acids. “Similarity” refers to the presence of not only identical amino acids but also the presence of conservative substitutions. The sequence similarity between two proteins is determined by aligning the residues of the two proteins (e.g., a candidate amino acid sequence and a reference amino acid sequence, such as Pn3Pase protein of the present disclosure, e.g., amino acids 41-765 of SEQ ID NO:2) to optimize the number of identical amino acids along the lengths of their sequences; gaps in either or both sequences are permitted in making the alignment in order to optimize the number of shared amino acids, although the amino acids in each sequence must nonetheless remain in their proper order. Sequence similarity may be determined, for example, using sequence techniques such as the BESTFIT algorithm in the GCG package (Madison WI), or the Blastp program of the BLAST 2 search algorithm, as described by Tatusova, et al. ( FEMS Microbiol Lett 1999, 174:247-250), and available through the World Wide Web, for instance at the internet site maintained by the National Center for Biotechnology Information, National Institutes of Health. Preferably, sequence similarity between two amino acid sequences is determined using the Blastp program of the BLAST 2 search algorithm. Preferably, the default values for all BLAST 2 search parameters are used. In the comparison of two amino acid sequences using the BLAST search algorithm, structural similarity is referred to as “identities.” Thus, reference to a protein described herein, such as amino acids 41-765 of SEQ ID NO:2, can include a protein with at least 80% identity, at least 81% identity, at least 82% identity, at least 83% identity, at least 84% identity, at least 85% identity, at least 86% identity, at least 87% identity, at least 88% identity, at least 89% identity, at least 90% identity, at least 91% identity, at least 92% identity, at least 93% identity, at least 94% identity, at least 95% identity, at least 96% identity, at least 97% identity, at least 98% identity, or at least 99% identity with the reference protein. Alternatively, reference to a protein described herein, such as amino acids 41-765 of SEQ ID NO:2, can include a protein with at least 80% similarity, at least 81% similarity, at least 82% similarity, at least 83% similarity, at least 84% similarity, at least 85% similarity, at least 86% similarity, at least 87% similarity, at least 88% similarity, at least 89% similarity, at least 90% similarity, at least 91% similarity, at least 92% similarity, at least 93% similarity, at least 94% similarity, at least 95% similarity, at least 96% similarity, at least 97% similarity, at least 98% similarity, or at least 99% similarity with the reference protein.

As used herein, “in vitro” refers to an artificial environment and to processes or reactions that occur within an artificial environment. In vitro environments can include, but are not limited to, a test tube. As used herein, the term “in vivo” refers to a natural environment that is within the body of a subject.

Conditions that “allow” an event to occur or conditions that are “suitable” for an event to occur, such as an enzymatic reaction, or “suitable” conditions are conditions that do not prevent such events from occurring. Thus, these conditions permit, enhance, facilitate, and/or are conducive to the event.

The term “and/or” means one or all of the listed elements or a combination of any two or more of the listed elements.

The words “preferred” and “preferably” refer to embodiments of the disclosure that may afford certain benefits, under certain circumstances. However, other embodiments may also be preferred, under the same or other circumstances. Furthermore, the recitation of one or more preferred embodiments does not imply that other embodiments are not useful, and is not intended to exclude other embodiments from the scope of the disclosure.

The terms “comprises” and variations thereof do not have a limiting meaning where these terms appear in the description and claims.

It is understood that wherever embodiments are described herein with the language “include,” “includes,” or “including,” and the like, otherwise analogous embodiments described in terms of “consisting of” and/or “consisting essentially of” are also provided.

Unless otherwise specified, “a,” “an,” “the,” and “at least one” are used interchangeably and mean one or more than one.

Also herein, the recitations of numerical ranges by endpoints include all numbers subsumed within that range (e.g., 1 to 5 includes 1, 1.5, 2, 2.75, 3, 3.80, 4, 5, etc.).

For any method disclosed herein that includes discrete steps, the steps may be conducted in any feasible order. And, as appropriate, any combination of two or more steps may be conducted simultaneously.

Reference throughout this specification to “one embodiment,” “an embodiment,” “certain embodiments,” or “some embodiments,” etc., means that a particular feature, configuration, composition, or characteristic described in connection with the embodiment is included in at least one embodiment of the disclosure. Thus, the appearances of such phrases in various places throughout this specification are not necessarily referring to the same embodiment of the disclosure. Furthermore, the particular features, configurations, compositions, or characteristics may be combined in any suitable manner in one or more embodiments.

The above summary of the present disclosure is not intended to describe each disclosed embodiment or every implementation of the present disclosure. The description that follows more particularly exemplifies illustrative embodiments. In several places throughout the application, guidance is provided through lists of examples, which examples can be used in various combinations. In each instance, the recited list serves only as a representative group and should not be interpreted as an exclusive list.

In the description herein particular embodiments may be described in isolation for clarity. Unless otherwise expressly specified that the features of a particular embodiment are incompatible with the features of another embodiment, certain embodiments can include a combination of compatible features described herein in connection with one or more embodiments.

### BRIEF DESCRIPTION OF THE FIGURES

The following detailed description of illustrative embodiments of the present disclosure may be best understood when read in conjunction with the following drawings.

FIG. 1 shows a repeating unit structure of Streptococcus pneumoniae serotype 3 capsular polysaccharide.

FIG. 2 shows a nucleotide sequence (SEQ ID NO:1) and amino acid (SEQ ID NO:2) of a full length Pn3Pase protein. The amino acids 41 and 765 are shown in underlined bold text

FIG. 3 shows an amino acid alignment of a truncated Pn3Pase protein, TM1, with the equivalent regions of five glycoside hydrolases from Paenibacillus. WP_079915027.1 (amino acids 1 - 765 of SEQ ID NO:2), amino acids 1-775 of WP_113018908.1 (SEQ ID NO:4), amino acids 1-759 of WP_068663733.1 (SEQ ID NO:5), amino acids 1-759 of WP_057315858.1 (SEQ ID NO:6), amino acids 1-759 of WP_056617367.1 (SEQ ID NO:8), and amino acids 1-759 of WP_028553222.1 (SEQ ID NO:9). The alignment was produced using the Clustal Omega multiple sequence alignment algorithm.

FIGS. 4A-C show characterization of Pn3Pase mutants. FIG. 4A) Description of Pn3Pase and derivatives. Extinction coefficients were determined using ExPASy ProtParam tool. FIG. 4B) Schematic representation of predicted domains (InterPro). Pn3Pase is full length native protein, WT is recombinant Pn3Pase, TM1,2,3 are truncation mutants (TM). CBM, carbohydrate binding module homologous to galactose binding domains. Signal peptide is represented by black rectangle. GH Family, region of homology to GH family 39; CBM, carbohydrate binding module; DUF 1080, region of homology to a β1,3-1,4 glucanase in GH family 16; Glucanase Do, region of homology to concanavalin A-like lectin/glucanase superfamily. FIG. 4C) Stain free SDS-PAGE gel of Pn3Pase mutants: lane 1, WT; lane 2, TM1; lane 3, TM2; lane 4, E196A; lane 5, E306A; lane 6, E196/306A; lane 7, TM3.

FIGS. 5A-C show activity characteristics of Pn3Pase mutants. PAHBAH assay was used to determine substrate degradation. All samples had 0.1 mg/ml Pn3P substrate and 30 nM (FIG. 5A), 60 nM (FIG. 5B), and 120 nM (FIG. 5C) enzyme incubated for 0, 30, 60 and 120 minutes. Legend for all graphs is the same and to the right of C. Significance for each data is determined using Student’s t test comparing data to corresponding time zero data.

FIG. 6 shows substrate saturation curve for WT Pn3Pase. Michaelis-Menton kinetics were determined plotting substrate concentration against initial velocities for WT Pn3Pase. For kinetics experiments WT received 1 µg/ml (5.9 nM) enzyme respectively. Substrate concentrations were 800, 400, 200, 100, 50, 25 and 0 nM. Initial velocities were determined as the slope of the line in amount of product formed between 0 and 8 minutes. Amount product formed was determined using pure tetrasaccharide solutions as standards. Kinetic parameters were determined using nonlinear regression (GraphPad Prism). Standard deviation of data was determined through independent duplicates.

FIGS. 7A-E show binding characterization of Pn3Pase mutants to Pn3P. Binding rates were determined using Biolayer interferometry (BLI). Biotinylated Pn3 polysaccharide (20 µg/mL) was attached to streptavidin coated sensor for 150 seconds then returned to baseline buffer. Sensor was then exposed to Pn3Pase or mutants (100 nm, 250 nm and 500 nm) for 300 seconds. Dissociation was measured over 300 seconds to determine K_{D}. K_{D}, kₒₙ and k_{off} rates were determined as the average of the three binding curves. All three binding curves are present for TM2 mutant, 100 nm and 250 nm curves are overlapping. Fits are shown as dotted lines.

FIG. 8 shows reaction mechanism of Pn3Pase. Region of the 600 MHz proton spectrum of Pn3P, [-3)-β-D-GlcA(1-4)-β-D-Glc(1-]ₙ in PBS buffer with 10% D2O at 37° C. identifying doublet signals from GlcA-HS. Bottom trace at time zero, additional spectra at times indicated.

FIG. 9 shows HMM search results against all HMMs for glycoside hydrolase families. The catalytic residues are underlined.

The schematic drawings are not necessarily to scale. Like numbers used in the figures refer to like components, steps and the like. However, it will be understood that the use of a number to refer to a component in a given figure is not intended to limit the component in another figure labeled with the same number. In addition, the use of different numbers to refer to components is not intended to indicate that the different numbered components cannot be the same or similar to other numbered components.

### DETAILED DESCRIPTION

### Proteins

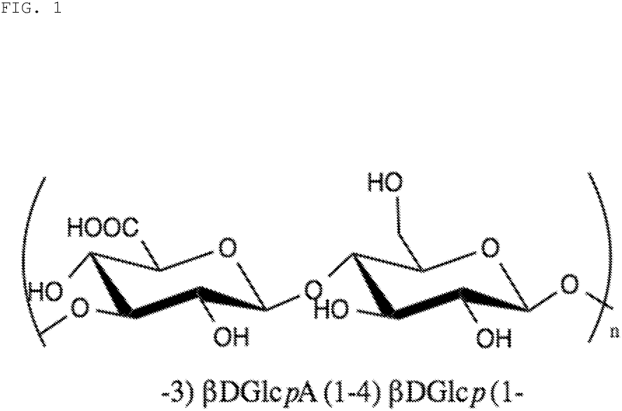

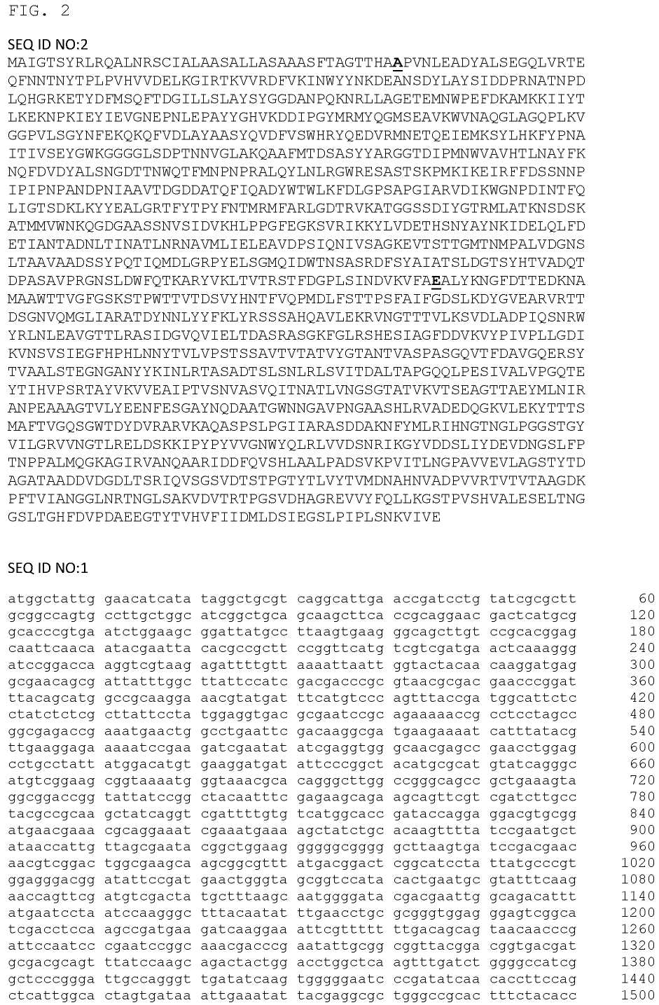

Provided herein are Pn3Pase proteins and methods for making and using the proteins. The Pn3Pase proteins described herein have Pn3Pase activity. A protein having Pn3Pase activity is referred to herein as a Pn3Pase protein or a Pn3Pase. A protein having Pn3Pase activity degrades type III capsular polysaccharide of Streptococcus pneumoniae. This capsular polysaccharide is also known as Pneumococcal type-3 polysaccharide (Pn3P) and is expressed by serotype 3 (also referred to as type 3) S. pneumoniae. The structure of Pn3P is shown in FIG. 1.

Whether a protein has Pn3Pase activity can be determined by in vitro assays. In one embodiment, an in vitro assay is carried out by measuring the increase in reducing ends using the p-hydroxybenzoic acid hydrazide method (see Example 1). Alternatively, Pn3P can be labeled with a detectable moiety, exposed to a protein being tested for Pn3Pase activity, and the reaction products resolved using a method that permits detection of differences in molecular weight. Pn3P can be obtained using methods that are known to the skilled person and routine or can be purchased from American Type Culture Collection, ATCC (Manassas, VA). Methods for detecting changes in molecular weight of polysaccharides are known to the skilled person and are routine.

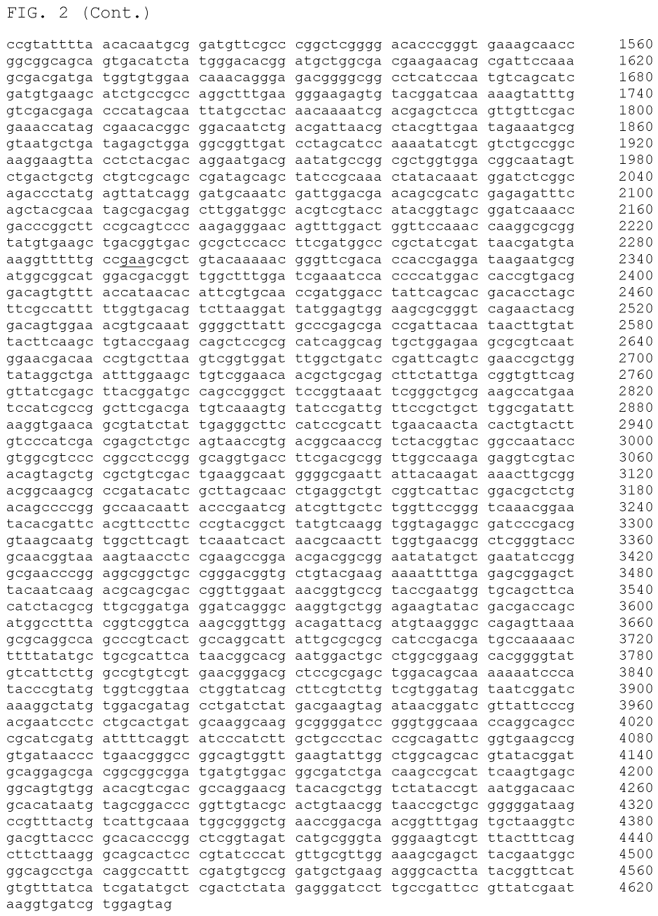

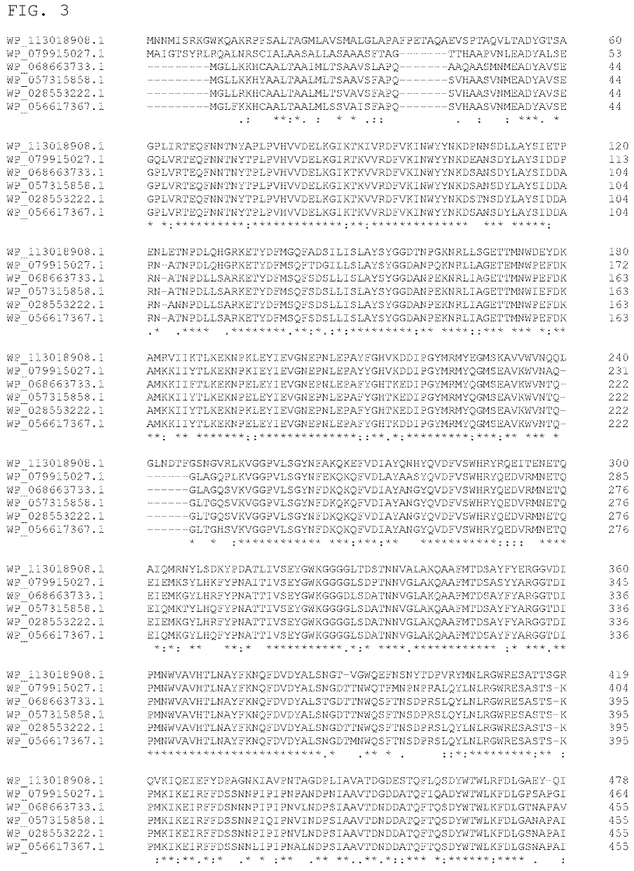

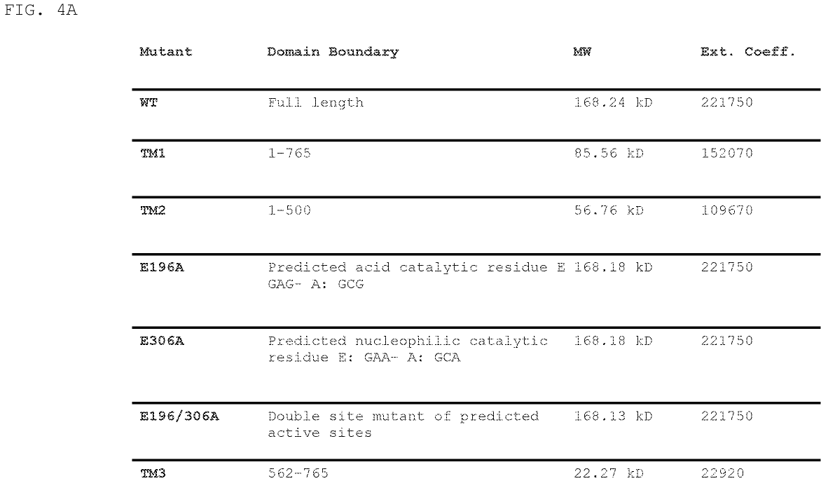

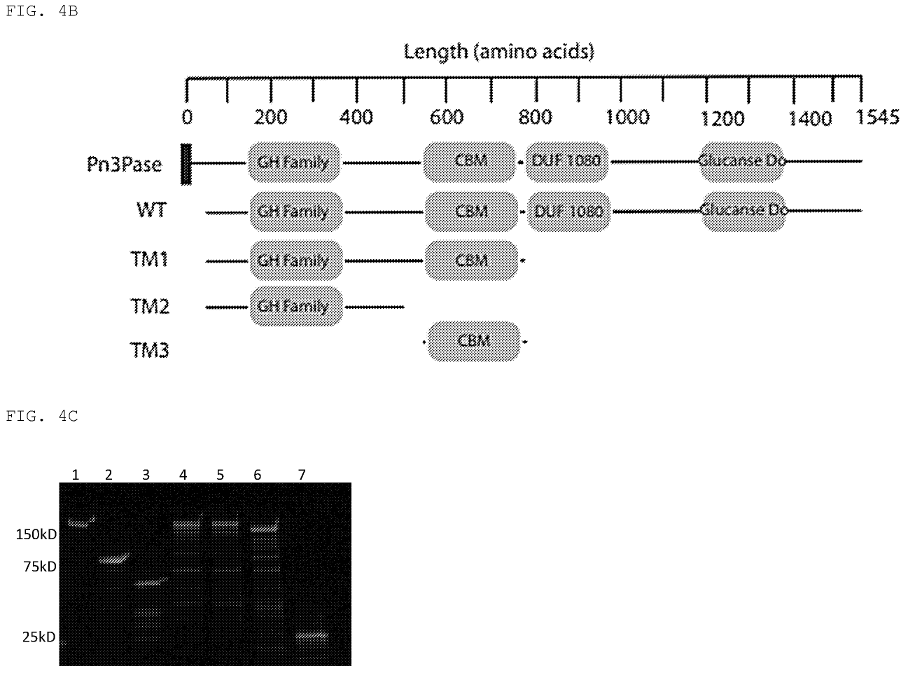

A Pn3Pase protein has been described (Avci et al., WO 2019/036373), and an example of a Pn3Pase is shown in FIG. 2 and designated SEQ ID NO:2. The inventors of the present disclosure have determined that Pn3Pase shows no significant sequence similarity to known glycoside hydrolase (GH) families over the entire length of a known catalytic domain, and that the enzymatic activity sites/domains, mechanism of action, substrate binding specificity, kinetic parameters, or structure were unknown. Sequence analysis of local similarities identified homology from amino acids 180 to 353 of SEQ ID NO:2 to a GH superfamily, and homology from amino acids 562 to 765 of SEQ ID NO:2 to galactose-binding-like superfamily, which the inventors determined as the potential carbohydrate binding module (CBM, see FIGS. 4). Site directed mutagenesis studies revealed two catalytic residues along with truncation mutants defining essential domains for function. Pn3Pase and its mutants were screened for activity, substrate binding and kinetics. This led to the establishment of a new glycoside hydrolase (GH) family of carbohydrate-active enzymes designated GH169, of which Pn3Pase is its founding member.

The Pn3Pase proteins of the present disclosure include truncation mutants of a full length Pn3Pase protein. Examples of a Pn3Pase protein of the present disclosure include the amino acids 41-765 of SEQ ID NO:2. Other examples of Pn3Pase proteins of the present disclosure include a fragment, e.g., those having fewer amino acids than amino acids 41-765 of SEQ ID NO:2. In one embodiment, a Pn3Pase protein of the present disclosure has fewer amino acids at the N-terminal end. For instance, any number of amino acids from residue 41 up to the region of homology to GH family 39, which begins at amino acid 180 of SEQ ID NO:2. Thus, in one embodiment a Pn3Pase protein of the present disclosure begins at amino acid 42, or at amino acid 43, or at amino acid 44, and so one up to beginning at amino acid 180.

Other examples of Pn3Pase proteins of the present disclosure include those having sequence similarity with the amino acids 41-765 of SEQ ID NO:2. The amino acid sequence of a Pn3Pase protein of the present disclosure having sequence similarity to amino acids 41-765 of SEQ ID NO:2 may include non-conservative or conservative substitutions of amino acids. In one embodiment, a substitution mutation is for a conservative amino acid. A conservative substitution is typically the substitution of one amino acid for another that is a member of the same class. For example, it is well known in the art of protein biochemistry that an amino acid belonging to a grouping of amino acids having a particular size or characteristic (such as charge, hydrophobicity, and/or hydrophilicity) may generally be substituted for another amino acid without substantially altering the secondary and/or tertiary structure of a protein. For the purposes of this disclosure, conservative amino acid substitutions are defined to result from exchange of amino acids residues from within one of the following classes of residues: Class I: Gly, Ala, Val, Leu, and Ile (representing aliphatic side chains); Class II: Gly, Ala, Val, Leu, Ile, Ser, and Thr (representing aliphatic and aliphatic hydroxyl side chains); Class III: Tyr, Ser, and Thr (representing hydroxyl side chains); Class IV: Cys and Met (representing sulfur-containing side chains); Class V: Glu, Asp, Asn and Gln (carboxyl or amide group containing side chains); Class VI: His, Arg and Lys (representing basic side chains); Class VII: Gly, Ala, Pro, Trp, Tyr, Ile, Val, Leu, Phe and Met (representing hydrophobic side chains); Class VIII: Phe, Trp, and Tyr (representing aromatic side chains); and Class IX: Asn and Gln (representing amide side chains). The classes are not limited to naturally occurring amino acids, but also include artificial amino acids, such as beta or gamma amino acids and those containing non-natural side chains, and/or other similar monomers such as hydroxyacids.

In one embodiment, a Pn3Pase protein of the present disclosure does not include a substitution of the glutamic acid (E) at residue 196, a substitution of the glutamic acid (E) at residue 306 of SEQ ID NO:2, or a substitution of the glutamic acid (E) at both residue 196 and residue 306 of SEQ ID NO:2. In one embodiment, a Pn3Pase protein of the present disclosure includes a conservative substitution of the glutamic acid (E) at residue 196 and/or residue 306 with an aspartic acid (Asp), asparagine (Asn), or glutamine (Gln) and maintains activity.

Guidance concerning how to make phenotypically silent amino acid substitutions is provided in Bowie et al. (1990, Science, 247:1306-1310), wherein the authors indicate proteins are surprisingly tolerant of amino acid substitutions. For example, Bowie et al. disclose that there are two main approaches for studying the tolerance of a protein sequence to change. The first method relies on the process of evolution, in which mutations are either accepted or rejected by natural selection. The second approach uses genetic engineering to introduce amino acid changes at specific positions of a cloned gene and selects or screens to identify sequences that maintain functionality. As stated by the authors, these studies have revealed that proteins are surprisingly tolerant of amino acid substitutions. The authors further indicate which changes are likely to be permissive at a certain position of the protein. For example, most buried amino acid residues require non-polar side chains, whereas few features of surface side chains are generally conserved. Other such phenotypically silent substitutions are described in Bowie et al, and the references cited therein.

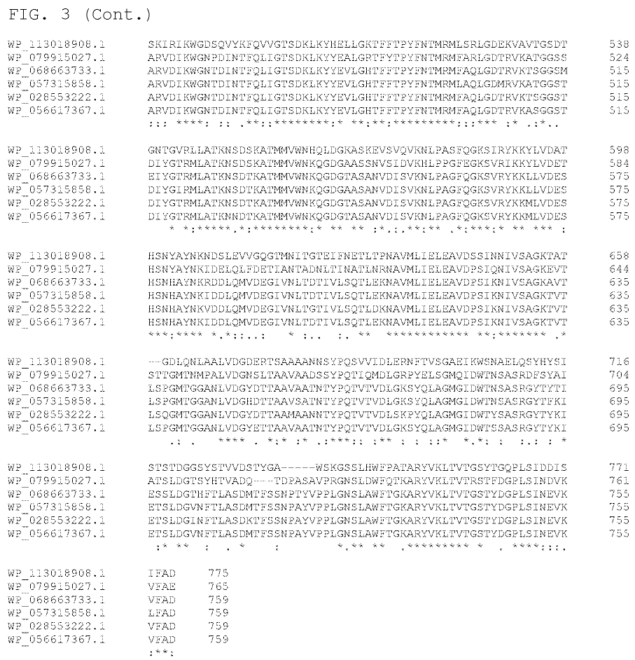

Guidance on how to modify the amino acid sequences of proteins disclosed herein is also provided at FIG. 3. FIG. 3 depicts a Clustal Omega amino acid alignment of the regions of structurally related glycoside hydrolase proteins expressed by Paenibacillus that correspond to amino acids 1-765 of SEQ ID NO:2. Clustal Omega is a multiple sequence alignment program (Sievers et al., 2011, Molecular Systems Biology 7: 539, doi:10.1038/msb.2011.75; Goujon et al., 2010, Nucleic acids research 38 (Suppl 2):W695-9, doi:10.1093/nar/gkq313). In FIG. 3 an asterisk (*) indicates positions which have a single, fully conserved residue; a colon (:) indicates conservation between groups of strongly similar properties, roughly equivalent to scoring > 0.5 in the Gonnet PAM 250 matrix; a period (.) indicates conservation between groups of weakly similar properties, roughly equivalent to scoring =< 0.5 and > 0 in the Gonnet PAM 250 matrix. By reference to this figure, the skilled person can predict which alterations to an amino acid sequence are likely to modify enzymatic activity, as well as which alterations are unlikely to modify enzymatic activity.

In one embodiment, a Pn3Pase protein of the present disclosure has the characteristic of displaying slightly better activity in sodium phosphate buffer at pH 7.2 than in MES buffer pH 6.0, and significantly worse in Tris buffer at pH 8.0 when compared to the full length Pn3Pase protein, for instance, SEQ ID NO:2 or amino acids 41-1545 of SEQ ID NO:2. In another embodiment, a Pn3Pase protein of the present disclosure displays a concentration-dependent preference for Ca2+, as it produces a higher concentration of reducing end GlcA in presence of 10 mM Ca2+ when compared to the full length Pn3Pase protein, for instance, SEQ ID NO:2 or amino acids 41-1545 of SEQ ID NO:2.

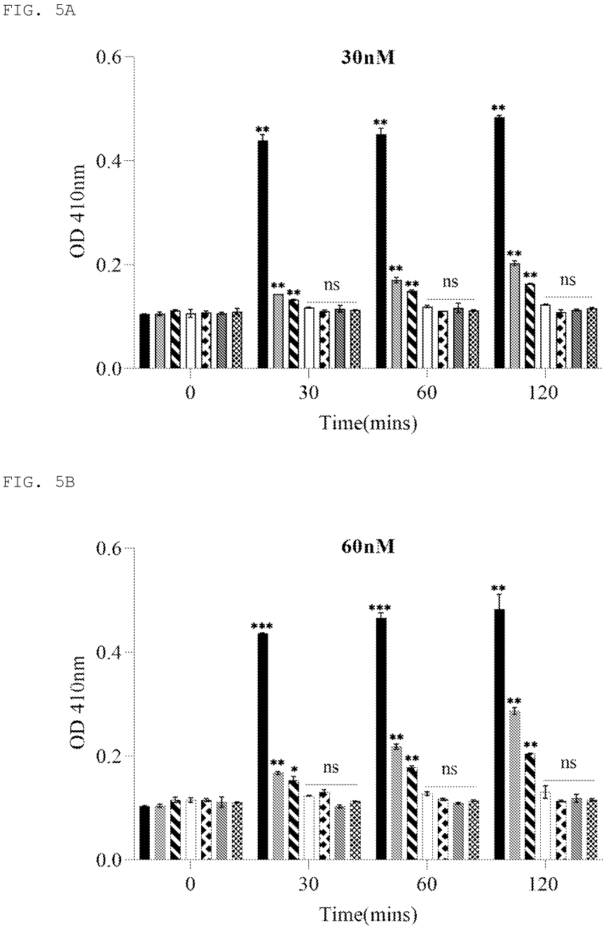

In one embodiment, a Pn3Pase protein of the present disclosure includes homologous amino acids at the N-terminal end, the C-terminal end, or both N-terminal end and C-terminal end. In one embodiment, the number of homologous amino acids at the N-terminal and/or C-terminal do not alter the better activity of the Pn3Pase protein described herein in sodium phosphate buffer at pH 7.2 than in MES buffer pH 6.0 when compared to the full length Pn3Pase protein, or the significantly worse activity of the PnPase protein described herein in Tris buffer at pH 8.0 when compared to the full length Pn3Pase protein, or the concentration-dependent preference for Ca2+ of the PnPase protein described herein when compared to the full length Pn3Pase protein, or the reduced activity of the PnPase protein described herein when compared to the full length Pn3Pase protein (see FIGS. 5). In one embodiment, a Pn3Pase protein of the present disclosure includes from 1 to 40 homologous amino acids at the N-terminal end, e.g., 1 (amino acid 40 of SEQ ID NO:2), 2 (amino acids 39 and 40 of SEQ ID NO:2 attached to the N-terminal end of the Pn3Pase protein), 3 (amino acids 38, 39, and 40 of SEQ ID NO:2 attached to the N-terminal end of the Pn3Pase protein), 4, 5, 6, 7, 8, 9, 10, and so on up to 40.

In one embodiment, a Pn3Pase protein of the present disclosure includes from 1 to 1545 homologous amino acids at the C-terminal end, e.g., 1 (amino acid 766 of SEQ ID NO:2), 2 (amino acids 766 and 767 of SEQ ID NO:2 attached to the C-terminal end of the Pn3Pase protein), 3 (amino acids 766, 767, and 768 of SEQ ID NO:2 attached to the C-terminal end of the Pn3Pase protein), 4, 5, 6, 7, 8, 9, 10, 20, 40, 60, 80, and so on up to 1545.

A Pn3Pase protein described herein can be expressed as a fusion protein that includes heterologous amino acids. For instance, the additional amino acid sequence may be useful for purification of the fusion protein by affinity chromatography. Amino acid sequences useful for purification can be referred to as a tag and include but are not limited to a polyhistidine-tag (His-tag) and maltose-binding protein. Representative examples may be found in Hopp et al. (U.S. Pat. No. 4,703,004), Hopp et al. (U.S. Pat. No. 4,782,137), Sgarlato (U.S. Pat. No. 5,935,824), and Sharma Sgarlato (U.S. Pat. No. 5,594,115). Various methods are available for the addition of such affinity purification moieties to proteins. Optionally, the additional amino acid sequence, such as a His-tag, can then be cleaved. In one embodiment, the additional amino acid sequence can include a cleavage site having sequence that can be identified by a protease and specifically cleaved. For example, a cleavage site can be one identified by a TEV protease.

In one embodiment, a Pn3Pase protein includes amino acids that act to target the Pn3Pase protein for secretion. The amino acids are typically referred to as a signal peptide or signal sequence. The signal peptide is typically present at the amino terminal end of the protein and is removed during transit of the protein to the exterior of the cell. The present disclosure is not limited by the signal peptide that can be present. Amino acid sequences that function as signal peptides are known in the art and are readily available. One non-limiting example is amino acids 1-40 of SEQ ID NO:2.

### Polynucleotides

Also provided herein are isolated polynucleotides encoding a Pn3Pase protein. A polynucleotide encoding a protein of the present disclosure having Pn3Pase activity is referred to herein as a Pn3Pase polynucleotide. Pn3Pase polynucleotides may have a nucleotide sequence encoding a protein having the amino acids 41-765 of SEQ ID NO:2. An example of the class of nucleotide sequences encoding such a protein is nucleotides 121-2295 of SEQ ID NO:1. It should be understood that a polynucleotide encoding a Pn3Pase protein represented by amino acids 41-765 of SEQ ID NO:2 is not limited to the nucleotide sequence disclosed at SEQ ID NO:1, but also includes the class of polynucleotides encoding such proteins as a result of the degeneracy of the genetic code. For example, the naturally occurring nucleotide sequence SEQ ID NO:1 is but one member of the class of nucleotide sequences encoding a protein having the amino acid sequence of residues 41-765 of SEQ ID NO:2. The class of nucleotide sequences encoding a selected protein sequence is large but finite, and the nucleotide sequence of each member of the class may be readily determined by one skilled in the art by reference to the standard genetic code, wherein different nucleotide triplets (codons) are known to encode the same amino acid.

A polynucleotide described herein may be present in a vector. A vector is a replicating polynucleotide, such as a plasmid, phage, or cosmid, to which another polynucleotide may be attached so as to bring about the replication of the attached polynucleotide. Construction of vectors containing a polynucleotide of the disclosure employs standard ligation techniques known in the art. See, e.g., Sambrook et al, Molecular Cloning: A Laboratory Manual., Cold Spring Harbor Laboratory Press (1989). A vector may provide for further cloning (amplification of the polynucleotide), i.e., a cloning vector, or for expression of the polynucleotide, i.e., an expression vector. The term vector includes, but is not limited to, plasmid vectors, viral vectors, cosmid vectors, and artificial chromosome vectors. Examples of viral vectors include, for instance, adenoviral vectors, adeno-associated viral vectors, lentiviral vectors, retroviral vectors, and herpes virus vectors. Typically, a vector is capable of replication in a microbial host, for instance, a microbe such as E. coli, or in a eukaryotic host, for instance, a yeast cell, a mammalian cell, or an insect cell. In one embodiment the vector is a plasmid.

Selection of a vector depends upon a variety of desired characteristics in the resulting construct, such as a selection marker, vector replication rate, and the like. In some aspects, suitable host cells for cloning or expressing the vectors herein include prokaryotic cells. Vectors may be introduced into a host cell using methods that are known and used routinely by the skilled person. For example, calcium phosphate precipitation, electroporation, heat shock, lipofection, microinjection, and viral-mediated nucleic acid transfer are common methods for introducing nucleic acids into host cells.

Polynucleotides encoding a truncated Pn3Pase protein described herein may be obtained from a microbe, for instance, Paenibacillus sp., or produced in vitro or in vivo. For instance, methods for in vitro synthesis include, but are not limited to, chemical synthesis with a conventional DNA/RNA synthesizer. Commercial suppliers of synthetic polynucleotides and reagents for such synthesis are well known.

An expression vector optionally includes regulatory sequences operably linked to the coding region. The disclosure is not limited by the use of any particular promoter, and a wide variety of promoters are known. Promoters act as regulatory signals that bind RNA polymerase in a cell to initiate transcription of a downstream (3′ direction) coding region. The promoter used may be a constitutive or an inducible promoter. It may be, but need not be, heterologous with respect to the host cell. The promoter useful in methods described herein may be, but is not limited to, a constitutive promoter, a temperature sensitive promoter, a non-regulated promoter, or an inducible promoter. In one embodiment, a promoter is one that functions in a member of the domain Bacteria. In one embodiment, a promoter is one that functions in a eukaryote.

An expression vector may optionally include a ribosome binding site and a start site (e.g., the codon ATG) to initiate translation of the transcribed message to produce the protein. It may also include a termination sequence to end translation. A termination sequence is typically a codon for which there exists no corresponding aminoacetyl-tRNA, thus ending protein synthesis. The polynucleotide used to transform the host cell may optionally further include a transcription termination sequence.

A vector introduced into a host cell to result in a genetically modified cell optionally includes one or more marker sequences, which typically encode a molecule that inactivates or otherwise detects or is detected by a compound in the growth medium. For example, the inclusion of a marker sequence may render the transformed cell resistant to an antibiotic, or it may confer compound-specific metabolism on the transformed cell. Examples of a marker sequence include, but are not limited to, sequences that confer resistance to kanamycin, ampicillin, chloramphenicol, tetracycline, streptomycin, and neomycin.

Proteins described herein may be produced using recombinant DNA techniques, such as an expression vector present in a cell. Such methods are routine and known in the art. The proteins may also be synthesized in vitro, e.g., by solid phase peptide synthetic methods. The solid phase peptide synthetic methods are routine and known in the art. A protein produced using recombinant techniques or by solid phase peptide synthetic methods may be further purified by routine methods, such as fractionation on immunoaffinity or ionexchange columns, ethanol precipitation, reverse phase HPLC, chromatography on silica or on an anion-exchange resin such as DEAE, chromatofocusing, SDS-PAGE, ammonium sulfate precipitation, gel filtration using, for example, Sephadex G-75, or ligand affinity.

### Genetically Modified Cells

Also provided is a genetically modified cell having a polynucleotide encoding a Pn3Pase described herein, e.g., amino acids 41-765 of SEQ ID NO:2. Compared to a control cell that is not genetically modified, a genetically modified cell can exhibit production of a Pn3Pase. A polynucleotide encoding a Pn3Psae may be present in the cell as a vector or integrated into genomic DNA, such as a chromosome or a plasmid, of the genetically modified cell. A cell can be a eukaryotic cell or a prokaryotic cell, such as a member of the domain Bacteria

Examples of host cells that are members of the domain Bacteria that can be genetically modified to include a polynucleotide encoding a Pn3Pase described herein include, but are not limited to, Escherichia (such as Escherichia coli), and Salmonella (such as Salmonella enterica, Salmonella typhi, Salmonella typhimurium).

Examples of host cells that are eukaryotic cells that can be genetically modified to include a polynucleotide encoding a Pn3Pase include, but are not limited to, yeast such as Saccharomyces cerevisiae and Pichia spp., insect cells, and mammalian cells.

### Compositions

Also provided are compositions that include a Pn3Pase protein described herein, e.g., amino acids 41-765 of SEQ ID NO:2, or a polynucleotide encoding a Pn3Pase protein described herein. Such compositions typically include a pharmaceutically acceptable carrier. As used herein “pharmaceutically acceptable carrier” includes saline, solvents, dispersion media, coatings, antibacterial and antifungal agents, isotonic and absorption delaying agents, and the like, compatible with pharmaceutical administration. Additional active agents can also be incorporated into the compositions.

A composition may be prepared by methods well known in the art of pharmaceutics. In general, a composition can be formulated to be compatible with its intended route of administration. Administration may be systemic or local. Examples of routes of administration include parenteral (e.g., intravenous, intradermal, subcutaneous, intraperitoneal, intramuscular), enteral (e.g., oral), and topical (e.g., epicutaneous, inhalational, transmucosal) administration. Appropriate dosage forms for enteral administration of the compound of the present disclosure include, but are not limited to, tablets, capsules or liquids. Appropriate dosage forms for parenteral administration may include intravenous or intraperitoneal administration. Appropriate dosage forms for topical administration include, but are not limited to, nasal sprays, metered dose inhalers, dry-powder inhalers or by nebulization.

Solutions or suspensions can include the following components: a sterile diluent such as water for administration, saline solution, fixed oils, polyethylene glycols, glycerin, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates; electrolytes, such as sodium ion, chloride ion, potassium ion, calcium ion, and magnesium ion, and agents for the adjustment of tonicity such as sodium chloride or dextrose. pH can be adjusted with acids or bases, such as hydrochloric acid or sodium hydroxide. Solutions or suspensions can include components that aid in increasing stability, increasing pharmacokinetics, and/or decreasing immunogenicity.

Compositions can include sterile aqueous solutions (where water soluble) or dispersions and sterile powders for the extemporaneous preparation of sterile solutions or dispersions. For parenteral administration, suitable carriers include physiological saline, bacteriostatic water, phosphate buffered saline (PBS), and the like. A composition is typically sterile and, when suitable for injectable use, should be fluid to the extent that easy syringability exists. It should be stable under the conditions of manufacture and storage and preserved against the contaminating action of microorganisms such as bacteria and fungi. The carrier can be a solvent or dispersion medium containing, for example, water, ethanol, polyol (for example, glycerol, propylene glycol, and liquid polyetheylene glycol, and the like), and suitable mixtures thereof. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as mannitol, sorbitol, sodium chloride in the composition. Prolonged absorption of the injectable compositions can be brought about by including in the composition an agent which delays absorption, for example, aluminum monostearate and gelatin.

Sterile solutions can be prepared by incorporating the active compound (e.g., a Pn3Pase protein described herein or a polynucleotide encoding the protein) in the required amount in an appropriate solvent with one or a combination of ingredients routinely used in pharmaceutical compositions, as required, followed by filtered sterilization. Generally, dispersions are prepared by incorporating the active compound into a sterile vehicle, which contains a basic dispersion medium and any other appropriate ingredients. In the case of sterile powders for the preparation of sterile injectable solutions, preferred methods of preparation include vacuum drying and freeze-drying which yields a powder of the active ingredient plus any additional desired ingredient from a previously sterilized solution thereof.

Oral compositions generally include an inert diluent or an edible carrier. For the purpose of oral therapeutic administration, the active compound can be incorporated with excipients and used in the form of tablets, troches, or capsules, e.g., gelatin capsules. Oral compositions can also be prepared using a fluid carrier. Pharmaceutically compatible binding agents and/or other useful materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation (e.g., topical administration), the active compounds can be delivered in the form of an aerosol spray from a pressured container or dispenser which contains a suitable propellant, e.g., a gas such as carbon dioxide, or a nebulizer.

Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

In those embodiments where a polynucleotide encoding a recombinant protein is administered, any method suitable for administration of polynucleotide agents can be used, such as gene guns, bio injectors, and skin patches as well as needle-free methods such as micro-particle DNA vaccine technologies (Johnston et al., U.S. Pat. No. 6,194,389).

The active compounds may be prepared with carriers that will protect the compound against rapid elimination from the body, such as a controlled release formulation, including implants. Biodegradable, biocompatible polymers can be used, such as ethylene vinyl acetate, polyanhydrides, polyglycolic acid, collagen, polyorthoesters, and polylactic acid. Such formulations can be prepared using standard techniques. Liposomal suspensions can also be used as pharmaceutically acceptable carriers. These can be prepared according to methods known to those skilled in the art.

Toxicity and therapeutic efficacy of the active compounds can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Recombinant proteins exhibiting high therapeutic indices are preferred.

The data obtained from cell culture assays and animal studies can be used in formulating a range of dosage for use in humans. The dosage of such compounds lies preferably within a range of circulating concentrations that include the ED50 with little or no toxicity. The dosage may vary within this range depending upon the dosage form employed and the route of administration used. For a compound used in the methods described herein, the therapeutically effective dose can be estimated initially from animal models. A dose may be formulated in animal models to achieve a circulating plasma concentration range that includes the IC50 (i.e., the concentration of the test compound which achieves a half-maximal inhibition of signs of disease, such as obesity). Such information can be used to more accurately determine useful doses in humans. Levels in plasma may be measured using routine methods.

The compositions can be administered one or more times per day to one or more times per week, including once every other day. The skilled artisan will appreciate that certain factors may influence the dosage and timing required to effectively treat a subject, including but not limited to the severity of the condition, previous treatments, the general health and/or age of the subject, and other diseases present. Moreover, treatment of a subject with an effective amount of an active compound can include a single treatment or, preferably, can include a series of treatments.

### Methods of Use

Also provided are methods. In one embodiment, a method is for making Pn3Pase protein described herein, e.g., amino acids 41-765 of SEQ ID NO:2. In one embodiment, the method includes incubating a genetically modified cell under suitable conditions for expression of a Pn3Pase protein. Optionally, the method includes introducing into a host cell a vector that includes a coding region encoding a Pn3Pase protein. In one embodiment, the method includes isolating or purifying the Pn3Pase protein from a cell or from a medium. In those embodiments where the Pn3Pase protein includes additional amino acids useful for isolating or purifying the protein, the method can also include cleavage of the additional amino acids from the Pn3Pase protein.

In one embodiment, a method is for cleaving a Pn3P molecule. In one embodiment, the method includes exposing a Pn3P molecule to a Pn3Pase protein. The P3nP molecule can be part of a Streptococcus pneumoniae microbe, e.g., the Pn3P molecule can be the capsular polysaccharide of an S. pneumoniae, or the Pn3P molecule can be separate from an S. pneumoniae microbe, e.g., the Pn3P molecule can be isolated. In one embodiment, the S. pneumoniae microbe is serotype 3. In one embodiment, the Pn3P molecule is in vivo, and in another embodiment, the Pn3P molecule is in vitro.

In one embodiment, a method is for reducing the amount of type III capsular polysaccharide on the surface of Streptococcus pneumoniae. The method includes contacting a Streptococcus pneumoniae having type III capsular polysaccharide present on its surface with a Pn3Pase protein. The Pn3Pase protein can be isolated or purified, and can be present in a composition. In one embodiment, the contacting can include exposing the microbe to a genetically modified cell that expresses the Pn3Pase protein. The contacting can be under conditions suitable for enzymatic hydrolysis of type III capsular polysaccharide. Optionally, the Streptococcus pneumoniae with the reduced amount of type III capsular polysaccharide has increased susceptibility to phagocytosis by macrophages, increased complement-mediated killing by neutrophils, or a combination thereof, compared to the Streptococcus pneumoniae that is not contacted with the Pn3Pase protein. In one embodiment, the S. pneumoniae microbe is serotype 3. In one embodiment, the S. pneumoniae microbe is in vivo, and in another embodiment, the S. pneumoniae microbe is in vitro.

In one embodiment, a method is for increasing deposition of at least one complement component on the surface of Streptococcus pneumoniae. The method includes contacting a Streptococcus pneumoniae having type III capsular polysaccharide present on its surface with a Pn3Pase protein. The Pn3Pase protein can be isolated or purified, and can be present in a composition. In one embodiment, the contacting can include exposing the microbe to a genetically modified cell that expresses the Pn3Pase protein. The contacting can be under conditions suitable for enzymatic hydrolysis of type III capsular polysaccharide. In one embodiment, the S. pneumoniae microbe is serotype 3. In one embodiment, the S. pneumoniae microbe is in vivo, and in another embodiment, the S. pneumoniae microbe is in vitro.

In one embodiment, a method includes treating an infection in a subject caused by Streptococcus pneumoniae. The subject used in a method described herein can be an animal such as, but not limited to, a murine (e.g., mouse or rat) or a human. The method includes administering an effective amount of the composition to an animal having an infection caused by Streptococcus pneumoniae. Optionally, the method can include determining whether the Streptococcus pneumoniae causing the infection has decreased. Methods for determining whether an infection is caused by a Streptococcus pneumoniae are routine and known in the art. The infection can be localized or systemic. An example of a localized Streptococcus pneumoniae infection is colonization of the nasal cavity, e.g., the nasopharynx. In one embodiment, topical administration of a composition described herein can be used to reduce nasopharyngeal colonization in a subject. Another example of a localized Streptococcus pneumoniae infection of the lung, e.g., pneumococcal pneumonia. In one embodiment, topical administration of a composition by, for instance, aerosol, can be used to reduce pneumococcal pneumonia in a subject. An example of a systemic Streptococcus pneumoniae infection is the presence of Streptococcus pneumoniae in the blood of a subject (e.g., bacteremia or sepsis). In one embodiment, parenteral administration of a composition can be used to reduce a systemic Streptococcus pneumoniae infection in a subject. In this aspect of the disclosure, an “effective amount” of a composition of the present disclosure is the amount able to elicit the desired response in the recipient, e.g., a reduction in the amount of Streptococcus pneumoniae present in a subject. The reduction can be a reduction in the number of S. pneumoniae in the nasopharynx, lung, or blood of a subject. The reduction can be a decrease of at least 2-fold, at least 3-fold, or at least 4-fold in the subject compared to the subject before administering the composition. In one embodiment, the S. pneumoniae microbe is serotype 3.

In another embodiment, a method includes treating one or more signs of certain conditions in animals that may be caused by infection by a Streptococcus pneumoniae. Streptococcuspneumoniae infections cause invasive pneumococcal disease. Examples of conditions caused by invasive pneumococcal disease include pneumonia, pneumococcal meningitis, otitis media, bacteremia, and sepsis. Signs associated with these conditions include chills, cough, rapid breathing, difficulty breathing, chest pain (pneumonia), stiff neck, fever, headache, confusion and photophobia (pneumococcal meningitis), and confusion, shortness of breath, elevated heart rate, pain or discomfort, over-perspiration, fever, and shivering (sepsis). In one embodiment, the S. pneumoniae microbe is serotype 3.

Treatment of one or more of these conditions can be prophylactic or, alternatively, can be initiated after the development of a condition described herein. Treatment that is prophylactic, for instance, initiated before a subject manifests a sign of a condition caused by Streptococcus pneumoniae, is referred to herein as treatment of a subject that is “at risk” of developing the condition. Typically, an animal “at risk” of developing a condition is an animal likely to be exposed to a Streptococcus pneumoniae causing the condition. Accordingly, administration of a composition can be performed before, during, or after the occurrence of the conditions described herein. Treatment initiated after the development of a condition may result in decreasing the severity of the signs of one of the conditions, including completely removing the signs. In this aspect of the disclosure, an “effective amount” is an amount effective to prevent the manifestation of signs of a condition, decrease the severity of the signs of a condition, and/or completely remove the signs. In one embodiment, the S. pneumoniae microbe is serotype 3.

The potency of a composition described herein can be tested according to standard methods. For instance, the use of mice as an experimental model for Streptococcus pneumoniae infection in humans is well established.

### Kits

The present disclosure also provides a kit for making a Pn3Pase protein. In one embodiment, the kit includes a vector that includes a coding region encoding a Pn3Pase protein described herein, e.g., amino acids 41-765 of SEQ ID NO:2, in an amount sufficient for transforming a cell. In one embodiment, the kit includes a genetically modified cell that includes a coding region encoding a Pn3Pase protein in a suitable packaging material.

In another embodiment, the present disclosure also provides a kit directed to using a Pn3Pase protein described herein. In one embodiment, the kit includes Pn3Pase protein, isolated or optionally purified, in a suitable packaging material.

Optionally, other reagents such as buffers or a pharmaceutically acceptable carrier (either prepared or present in its constituent components, where one or more of the components may be premixed or all of the components may be separate), and the like, are also included. In one embodiment, the protein, vector, or genetically modified cell may be present with a buffer, or may be present in separate containers. Instructions for use of the packaged components are also typically included.

As used herein, the phrase “packaging material” refers to one or more physical structures used to house the contents of the kit. The packaging material is constructed by known methods, preferably to provide a sterile, contaminant-free environment. The packaging material has a label, which indicates that the contents can be used for transforming a cell or producing Pn3Pase protein. In addition, the packaging material contains instructions indicating how the materials within the kit are used. As used herein, the term “package” refers to a solid matrix or material such as glass, plastic, paper, foil, and the like, capable of holding within fixed limits a vector or a genetically modified cell. Thus, for example, a package can include a glass or plastic vial used to contain appropriate quantities of a Pn3Pase protein. “Instructions for use” typically include a tangible expression describing the reagent concentration or at least one method parameter.

The invention is defined in the claims. However, below there is provided a non-exhaustive listing of non-limiting exemplary aspects. Any one or more of the features of these aspects may be combined with any one or more features of another example, embodiment, or aspect described herein.

### Exemplary Aspects

Aspect 1 is a non-natural Pn3Pase protein, also referred to as a truncated Pn3Pase protein, comprising an amino acid sequence of at least 80% identity with amino acids 41-765 of SEQ ID NO:2. In one embodiment, the protein can include an aspartic acid (Asp), asparagine (Asn), or glutamine (Gln) at position 196, position 306, or a combination thereof.

Aspect 2 is the protein of Aspect 1 further comprising at least one heterologous amino acid at the amino-terminal end, the carboxy-terminal end, or both amino- and carboxy-terminal ends.

Aspect 3 is the protein of Aspect 1 or 2 wherein the protein consists of amino acids 41-765 of SEQ ID NO: 1.

Aspect 4 is the protein of any one of Aspects 1-3 wherein the heterologous amino acids comprise a tag.

Aspect 5 is a polynucleotide comprising a coding region, wherein the coding region comprises a nucleotide sequence encoding the protein of any one of Aspects 1-4.

Aspect 6 is the polynucleotide of Aspect 5 wherein the polynucleotide is present in a vector.

Aspect 7 is a genetically modified cell comprising the protein of any one of Aspects 1-6.

Aspect 8 is a genetically modified cell comprising an exogenous polynucleotide comprising a coding region, wherein the coding region comprises a nucleotide sequence encoding the protein of any one of Aspects 1-7.

Aspect 9 is the genetically modified cell of Aspect 7 or 8 wherein the cell is a eukaryotic cell.

Aspect 10 is the genetically modified cell of any one of Aspects 7-9 wherein the cell is a mammalian cell, a yeast cell, or an insect cell.

Aspect 11 is the genetically modified cell of any one of Aspects 7-10 wherein the cell is a prokaryotic cell.

Aspect 12 is the genetically modified cell of any one of Aspects 7-11 wherein the cell is E. coli.

Aspect 13 is a composition comprising the non-natural P3nPase protein of any one of Aspects 1-4.

Aspect 14 is a composition comprising the genetically modified cell of any one of Aspects 7-12.

Aspect 15 is a composition comprising the polynucleotide of Aspect 5 or 6.

Aspect 16 is the composition of any one of Aspects 13-15 wherein the composition comprises a pharmaceutically acceptable carrier.

Aspect 17 is a method comprising incubating (i) a cell comprising the polynucleotide of claim 5, or (ii) the genetically modified cell of any one of Aspects 7-12 under conditions suitable for expression of the protein.

Aspect 18 is the method of Aspect 17 further comprising isolating the protein.

Aspect 19 is the method of Aspect 17 or 18 further comprising purifying the protein.

Aspect 20 is the method of any one of Aspects 17-19 wherein the cell is a eukaryotic cell.

Aspect 21 is the method of any one of Aspects 17-20 wherein the cell is a mammalian cell, a yeast cell, or an insect cell.

Aspect 22 is the method of any one of Aspects 17-19 wherein the cell is a prokaryotic cell.

Aspect 23 is the method of any one of Aspects 17-19 or 22 wherein the prokaryotic cell is E. coli.

Aspect 24 is a method comprising contacting a type III capsular polysaccharide with the protein of any one of Aspects 1-4, wherein the contacting is under conditions suitable for enzymatic hydrolysis of type III capsular polysaccharide.

Aspect 25 is a method comprising contacting a Streptococcus pneumoniae comprising a type III capsular polysaccharide with the non-natural Pn3Pase protein of any one of Aspects 1-4, wherein the contacting is under conditions suitable for enzymatic hydrolysis of type III capsular polysaccharide, wherein the amount of type III capsular polysaccharide on the surface of the S. pneumoniae is reduced compared to the S. pneumoniae that is not contacted with the Pn3Pase protein.

Aspect 26 is a method for increasing deposition of at least one complement component on the surface of a Streptococcus pneumoniae, the method comprising contacting a Streptococcus pneumoniae comprising a type III capsular polysaccharide with the non-natural Pn3Pase protein of any one of Aspects 1-4, wherein the deposition of at least one complement component on the surface of the S. pneumoniae is increased compared to the S. pneumoniae that is not contacted with the Pn3Pase protein.

Aspect 27 is the method of any one of Aspects 24-26 wherein the S. pneumoniae is present in conditions suitable for replication of the S. pneumoniae.

Aspect 28 is the method of any one of Aspects 24-27 wherein the contacting comprises exposing the type III capsular polysaccharide or the S. pneumoniae to a genetically modified cell that expresses the non-natural Pn3Pase protein.

Aspect 29 is the method of any one of Aspects 24-27 wherein the S. pneumoniae has increased susceptibility to phagocytosis by macrophages, increased complement-mediated killing by neutrophils, or a combination thereof, compared to the S. pneumoniae that is not contacted with the Pn3Pase protein.

Aspect 30 is a method for treating an infection in a subject, the method comprising administering an effective amount of the composition of any one of Aspects 13-16 to a subject having or at risk of having an infection caused by a serotype 3 S. pneumoniae.

Aspect 31 is a method for treating a sign in a subject, the method comprising administering an effective amount of the composition of any one of Aspects 13-16 to a subject having or at risk of having an infection caused by a serotype 3 S. pneumoniae.

Aspect 32 is a method for decreasing colonization in a subject, the method comprising administering an effective amount of a composition comprising the composition of any one of Aspects 13-16 to a subject colonized by or at risk of being colonized by a serotype 3 S. pneumoniae.

Aspect 33 is the method of any one of Aspects 30-32 wherein the subject is a human.

Aspect 34 is a non-natural Pn3Pase protein disclosed herein for use in therapy, for use as a medicament, for use in the treatment of a condition, or a combination thereof.

Aspect 35 is a use of a non-natural Pn3Pase protein disclosed herein for preparation of a medicament for the treatment of pneumonia, pneumococcal meningitis, otitis media, bacteremia, sepsis, or a combination thereof.

Aspect 36 is a composition comprising a non-natural P3nPase protein described herein for use in treating or preventing an infection or a sign caused by a serotype 3 S. pneumoniae.

### EXAMPLES

The present disclosure is illustrated by the following examples. It is to be understood that the particular examples, materials, amounts, and procedures are to be interpreted broadly in accordance with the scope and spirit of the disclosure as set forth herein.

### Example 1

Characterization of the β-glucuronidase Pn3Pase as the founding member of glycoside hydrolase family GH169

### Introduction

A number of early studies demonstrated an enzyme found in Bacillus circulans was capable of degrading the capsular polysaccharide of highly virulent type 3 Streptococcus pneumoniae (Pn3P) (Avery, O.T. and Dubos, R. 1930, Dubos, R. and Avery, O.T. 1931, Sickles, G.M. and Shaw, M. 1934). However, upon sequencing the genome of this bacterial strain “Bacillus circulans Jordan strain 32352” we discovered this bacterium rather belonged within the Paenibacillus genus and was reestablished as Paenibacillus sp. 32352 (Middleton, D.R., Lorenz, W., et al. 2017). As previously reported, this soil-dwelling Paenibacillus bacterium is capable of producing a glycoside hydrolase (GH), Pn3Pase, which degrades the linear repeating disaccharide polymer -3)βGlcA(1-4)βGlc(1-that makes up Pn3P (Avery, O.T. and Dubos, R. 1930, Dubos, R. and Avery, O.T. 1931, Sickles, G.M. and Shaw, M. 1934). Recently, we reported further on Pn3Pase identifying and cloning the Pn3Pase gene from Paenibacillus sp. 32352, determining optimal activity parameters and characterizing oligosaccharide products from Pn3P degradation (Middleton, D.R., Zhang, X., et al. 2018). Further, we have reported on Pn3Pase potential as a therapeutic agent against type 3 pneumococcal infections (Middleton, D.R., Paschall, A.V., et al. 2018).

With the reclassification of the Paenibacillus bacteria and exploration into the Pn3Pase gene it appeared this glycoside hydrolase did not belong to any existing carbohydrate-active enzyme (CAZy) (Lombard, V., Golaconda Ramulu, H., et al. 2014) (http://www.cazy.org) family. Sequence alignments revealed no significant homology to current CAZy GH families over the entire length of a known catalytic domain; however, InterPro analysis suggests local similarities to GH family 39 (Mitchell, A.L., Attwood, T.K., et al. 2019). Additionally, Phyre2 analysis suggests sequence similarities to glucuronidases (Kelley, L.A., Mezulis, S., et al. 2015). Sequence alignments also suggest Pn3Pase may belong to the GH-A clan and thus may function with a retaining mechanism, as other GH-A clan members. Further, apart from this enzyme being sequentially unique it is also functionally unique. To our knowledge, this is the only enzyme that demonstrates Pn3P hydrolysis activity. Taken together, this information suggested Pn3Pase may establish and belong to a new CAZy GH family.

Given that Pn3Pase has potential as a therapeutic agent against the highly infectious serotype 3 S. pneumoniae, determining the structure function relationship of this enzyme is crucial to generating Pn3Pase derivatives with optimal activity and pharmacokinetics. Apart from the study exploring Pn3Pase as a therapeutic (Middleton, D.R., Paschall, A.V., et al. 2018) and the work on elucidating the Pn3Pase gene for recombinant protein expression (Middleton, D.R., Zhang, X., et al. 2018) little work has been done on this enzyme. We do not yet know enzymatic activity sites/domains, mechanism of action, substrate binding specificity, kinetic parameters, or structure.

In continuation of our work characterizing this important enzyme, here we present the detailed biochemical analysis of Pn3Pase. This work led to the establishment of a new GH family designated GH169 of which Pn3Pase is its founding member. Site directed mutagenesis revealed the nucleophile and acid/base catalytic residues, while C terminal truncations indicated domains crucial for activity. Binding and kinetic assays revealed this enzyme’s activity towards Pn3P and its substrate specificity. NMR analysis demonstrated Pn3Pase utilizes a retaining mechanism similar to other GH-A clan members. Taken together, this work provides the biochemical basis for the Pn3P hydrolysis catalyzed by Pn3Pase. Additionally, this work will aid in developing Pn3Pase as a therapeutic agent against the highly pathogenic type 3 pneumococcus infection.

### Results

### Pn3Pase Domain Analysis and Production

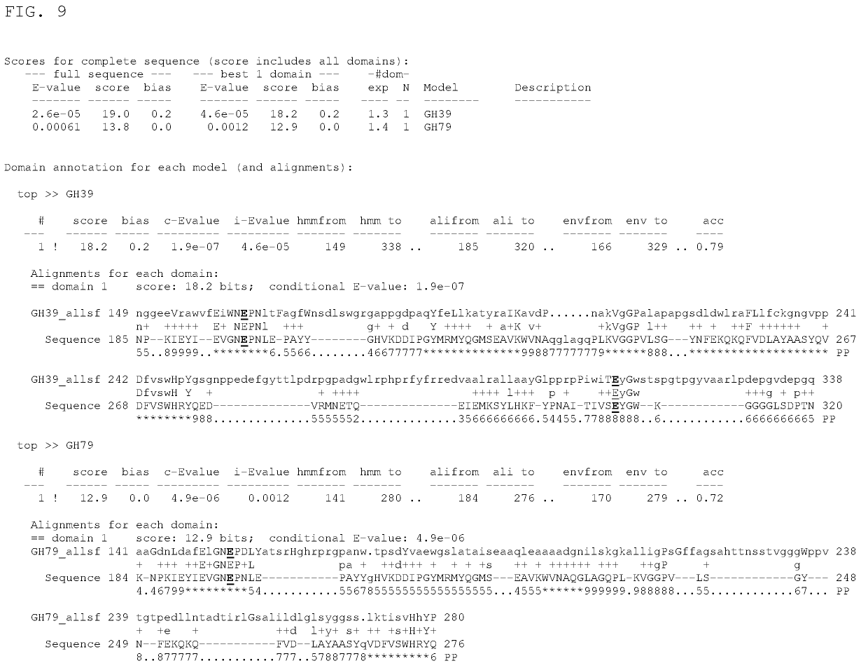

We previously reported on the Paenibacillus sp. 32352 gene, Pbac_3551, which encodes for the Pn3Pase protein (Middleton, D.R., Zhang, X., et al. 2018). The translated protein sequence of Pn3Pase contains 1545 amino acids and yields a mature protein of 164.1 kDa. InterPro sequence analysis (Mitchell, A.L., Attwood, T.K., et al. 2019) of the full protein revealed putative domains (FIGS. 4A,B). There was homology between residues 180 and 353 to glycoside hydrolase superfamily, with suggested homology to GH family 39 at the N-terminal region. Other predicted regions of interests were homology to galactose-binding-like superfamily, which for the purpose of this study we took to be the potential carbohydrate binding module (CBM) (FIG. 4B), a domain of unknown function 1080 (DUF 1080), which has structural similarity to a β1,3-1,4 glucanase in GH family 16, and a concanavalin A-like lectin/glucanase superfamily (FIG. 4B). Alignments with existing GHs in the CAZy database revealed short segments of homology with carbohydrate-active enzymes; however, no significant homology across the full length of Pn3Pase exists. Pn3Pase was run against CAZy HMMs built for GH families (HMMer2,3) which yielded scores insufficient for inclusion in any existing CAZy GH family. The only two borderline hits, GH39 and GH79, showed local similarity that did not extend along the length of the catalytic domains. However, the alignments with GH39 and GH79 showed conservation of the subsequence surrounding the known acid/base residue of clan GH-A (E in GNEPN) and the known nucleophile (E in VSEYGW), with the later only visible in the alignment with GH39 (FIG. 9) (Henrissat, B., Callebaut, I., et al. 1996). Altogether these low scores and incomplete alignments suggested Pn3Pase would establish a new GH family related to clan GH-A, have potential catalytic residues at amino acid sites 196 and 306, and suggested Pn3Pase works through a retaining mechanism. Enzymes belonging to clan GH-A function through retaining mechanisms with their catalytic domain having a β/α₈ barrel fold (Henrissat, B. and Bairoch, A. 1996) suggesting Pn3Pase may also have these features.

Moving forward with this preliminary evidence, we performed site directed mutagenesis and deletion studies to confirm catalytic residues and domains important for function (FIGS. 4). FIGS. 4A and 4B describe the Pn3Pase derivatives and their predicted domains used in this study. WT Pn3Pase and mutant coding sequences were amplified and cloned into pET-DEST42 vector with a C-terminal His₆tag, and expressed in E. coli. Recombinant proteins were purified using affinity chromatography followed by size exclusion chromatography and purity was assessed using SDS-PAGE gel (FIG. 4C). Bands at appropriate molecular weights (FIG. 4A) for each protein were observed (FIG. 4C). Oligonucleotides used to generate the mutants are displayed in Table 1 and site directed mutants were confirmed through sequencing.

TABLE 1

Oligonucleotides for each protein used in this study

| | |
|---|---|
| Oligonucleotide | Sequence 5′- 3 |
| WT_F | ggggacaagtttgtacaaaaaagcaggcttcgaaggagatagaaccatggcacccgtgaatctggaagc (SEQ ID NO:10) |
| WT_R | ggggaccactttgtacaagaaagctgggtgctccacgatcaccttattcgataacg (SEQ ID NO:11) |
| TM1_R | ggggaccactttgtacaagaaagctgggtgttcggcaaaaacctttacatcg (SEQ ID NO:12) |
| TM2_R | ggggaccactttgtacaagaaagctgggtgggtgtagaaagtgcggcc (SEQ ID NO:13) |
| E196A_F | gggcaacgcgccgaacc (SEQ ID NO:14) |
| E196A_R | ggttcggcgcgttgccc (SEQ ID NO:15) |
| E306A_F | tgttagcgcatacggctggaag (SEQ ID NO:16) |
| E306A_R | cttccagccgtatgcgctaaca (SEQ ID NO:17) |
| TM3_F | ggggacaagtttgtacaaaaaagcaggcttcgaaggagatagaaccatgaatatcgtgtctgccggc (SEQ ID NO:18) |
| TM3_R | ggggaccactttgtacaagaaagctgggtgttcggcaaaaacctttacatcg (SEQ ID NO:19) |

### Biochemical Characterization of Pn3Pase Mutants

To assess domains important for function and predicted catalytic residues, we performed activity assays utilizing the p-hydroxybenzoic acid hydrazide (PAHBAH) method (Blakeney, A.B. and Mutton, L.L. 1980). This method labels the reducing ends of sugars and would reveal if Pn3P hydrolysis was occurring through each Pn3Pase derivative. Unmodified Pn3Pase and its derivatives were first assayed under previously established conditions utilizing 10 µg/mL Pn3P substrate in PBS with 30 nM enzyme for 30-120 minutes (FIG. 5A). As expected, WT reached Pn3P hydrolysis saturation within 30 minutes with labeled reducing ends not significantly increasing at 60 or 120 minutes (FIG. 5A). Interestingly, TM1 and E196A mutant began displaying significant hydrolysis activity at 30 minutes which continued to increase over the time course. However, no other Pn3Pase mutant showed hydrolase activity even after 4 hours (FIG. 5A). To determine if these derivatives truly exhibited no activity against Pn3P we increased the enzyme concentration to 2× and 4× that of the original concentration (FIGS. 5B,C). Again, no derivatives except TM1 and E196A displayed Pn3P hydrolase activity even at increased enzyme concentrations. TM1 displayed increased levels of activity compared to E196A, which was especially evident at 120 nM where TM1 reached hydrolysis levels similar to WT after 120 minutes (FIG. 5C).

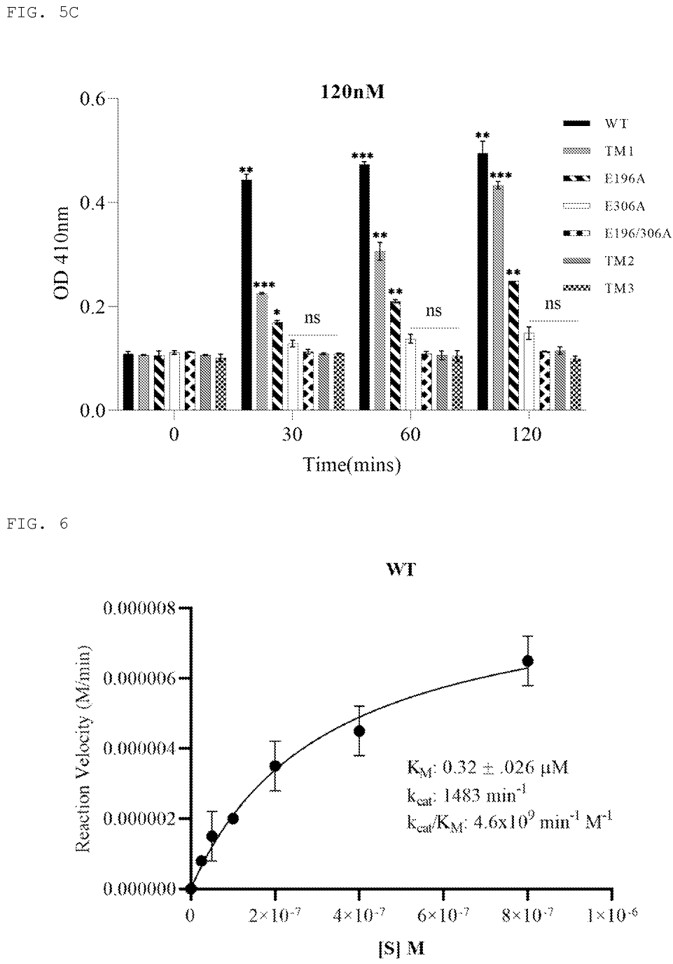

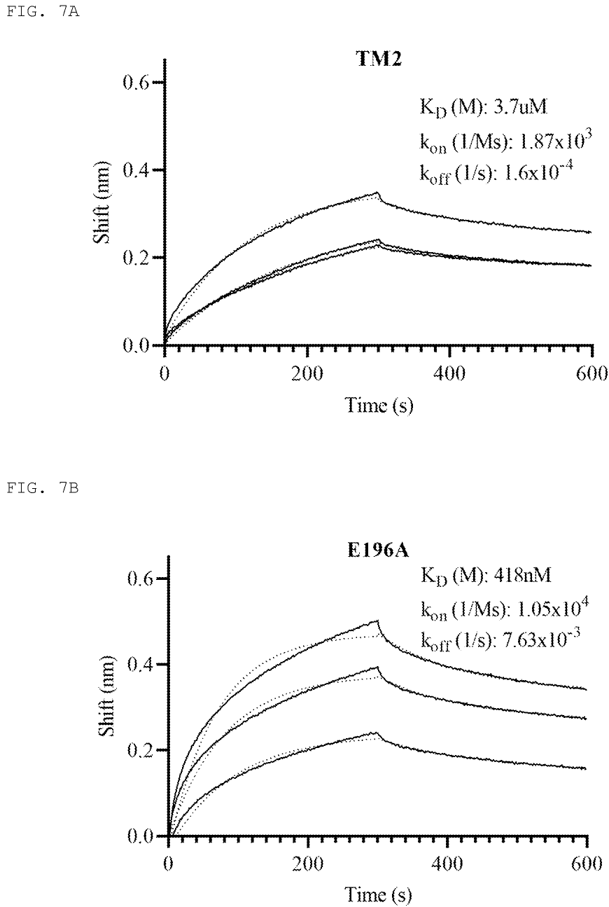

To continue the biochemical assessment of Pn3Pase activity, we measured Michaelis-Menten kinetic parameters utilizing PAHBAH assay. Since WT was the only enzyme to display significant activity levels at early time points with low enzyme and substrate levels we only carried on with this protein. Michaelis-Menten parameters of WT Pn3Pase were determined using different concentrations of Pn3P substrate ranging from 0-800 µM with 1 µg/mL (5.9 nM) enzyme. WT Pn3Pase displayed activity kinetics with a k_{cat} of 1483 min⁻¹ and K_{M} of 0.32 ± 0.026 µM (FIG. 6). The catalytic efficiency defined by the specificity constant k_{cat/}K_{M} was calculated as 4.6×10⁹ min⁻¹M⁻¹.

Taken together, these results suggest that the glutamic acid residues at 196 and 306 positions function as catalytic residues. Further, both E196 and E306 are conserved glutamic acids in Pn3Pase and its close homologs as is the case for clan GH-A glycoside hydrolases (Davies, G. and Henrissat, B. 1995) making them the acid/base and the nucleophilic residues respectively. Additionally, amino acids and domains after residue 765 do not appear to be crucial for function, while the putative carbohydrate binding module is required for function. While TM1 and E196A are the only Pn3Pase mutants to display function, they hydrolyze at reduced efficiency compared to WT Pn3Pase.

### Pn3Pase Binding Affinity to Pn3P

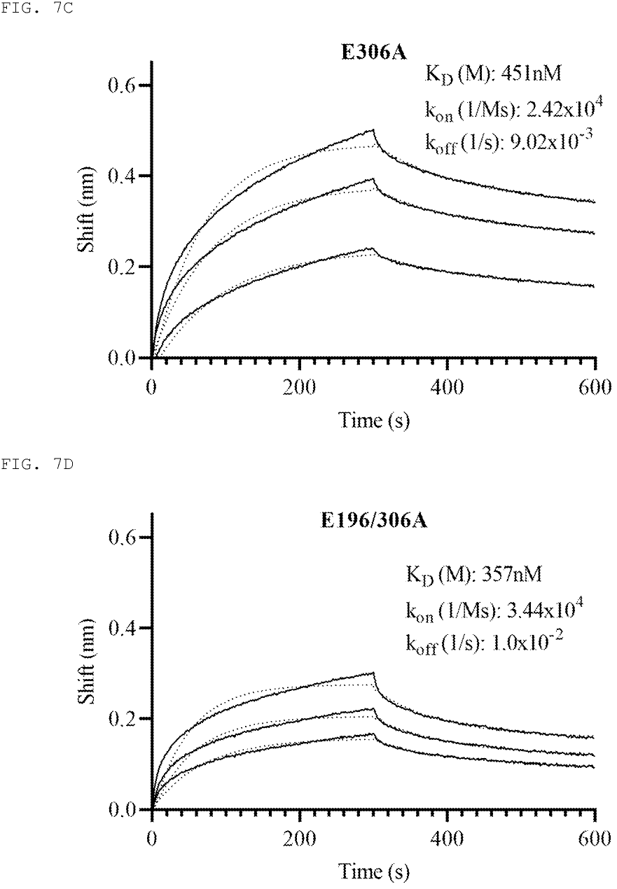

To determine Pn3Pase binding affinity for its substrate we utilized biolayer interferometry. Pn3P was biotinylated, bound to streptavidin biosensors and Pn3Pase proteins were used as ligand (FIGS. 7). We used only the non-catalytic Pn3Pase (low catalytic activity for E196A) mutants for this assay as WT and TM1 significantly hydrolyzed the substrate during the course of the experiment and therefore showed little to no binding (data not shown). The site mutants E196A, E306A, and E196/306A had high affinities with K_{D} (equilibrium dissociation constant) of 418 nM, 451 nM, and 357 nM respectively. TM2 binding curves at 100 nM and 250 nM are both present but overlapped and appeared as a single curve. TM2 had a K_{D} roughly 10-fold higher than site mutants at 3.7 µM which appears to be due to a slower kₒₙ (second-order rate constant of the binding reaction) rate. This is to be expected as TM2 mutant lacks the putative CBM which has been suggested to play a large role in substrate binding (Klontz, E.H., Trastoy, B., et al. 2019). Importantly, this previous study also suggests both the glycoside hydrolase and carbohydrate binding modules are involved in binding elucidating why we observed binding even in the absence of a CBM albeit at a lower affinity than non-catalytic full length mutants (Klontz, E.H., Trastoy, B., et al. 2019). In addition, we observed no apparent binding of TM3 to Pn3P substrate (FIGS. 7). As TM3 is the putative carbohydrate binding module mutant we expected to see binding; however, it has been suggested some CBMs display weak binding interactions as could be the case for our protein (Volkov, I.I.u., Lunina, N.A., et al. 2004).

### Pn3Pase Reaction Mechanism

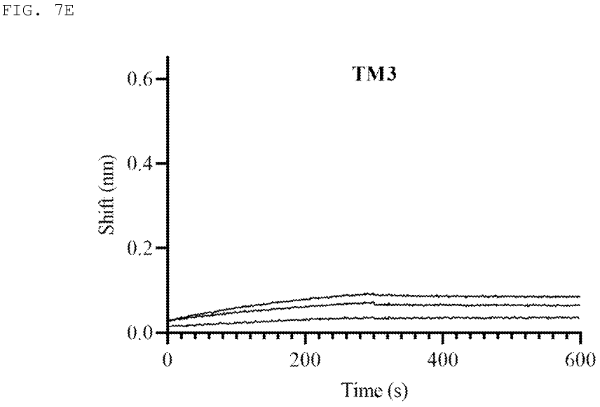

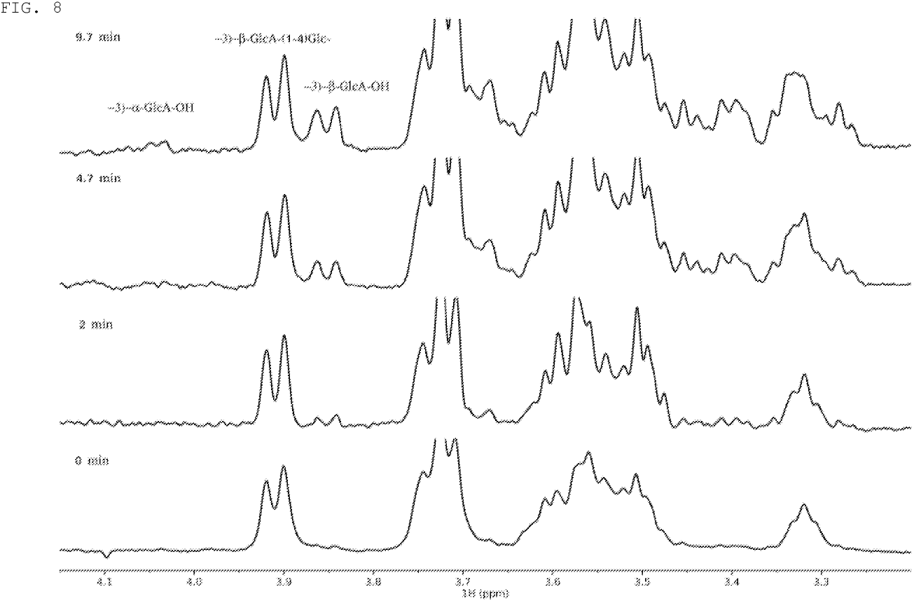

Next, we determined whether Pn3Pase acts through a mechanism that yields either a retention or inversion of the stereochemistry at the GlcA anomeric reducing end (FIG. 8). We used ¹H NMR spectroscopy to monitor the hydrolysis of the β-GlcA(1-4)β-Glc-glycosidic linkage in Pn3P by Pn3Pase over time. Chemical shifts of Pn3P were based on previously published data (Middleton, D.R., Zhang, X., et al. 2018). Since the β-H1 signal of free reducing GlcA (4.60 ppm) is obscured by the overlapping water signal at 37° C. and further reduced by the water suppression NMR pulse program, the GlcA H5 signals were used to monitor the reaction (FIG. 8). The Pn3P starting material shows the glycosidically linked β-GlcA H5 signal at 3.9 ppm (bottom trace, FIG. 8). Immediately after the addition of enzyme a signal corresponding to the H5 of the free reducing β-anomer of GlcA (3.85 ppm) is produced and continues to grow over time (FIG. 8). Only after around 10 minutes, does a small signal appear corresponding to the H5 of the free reducing α-GlcA (4.04 ppm) (FIG. 8, top trace), which is due to mutarotation. This confirms that Pn3Pase functions through a retaining mechanism and supports an activity mechanism that functions through a nucleophile and an acid/base catalytic residues (Davies, G. and Henrissat, B. 1995) as other clan GH-A members further suggesting Pn3Pase may belong to this GH clan. Taken together these results demonstrate that Pn3Pase acts via a retaining mechanism to hydrolyze Pn3P.

### Discussion

The capsular polysaccharides (CPS) of many pathogenic bacteria are prominent features serving many functions such as assisting in adhesion and colonization and inhibition of opsonophagocytosis rendering them major virulence factors (Magee, A.D. and Yother, J. 2001, Moxon, E.R. and Kroll, J.S. 1990). Unencapsulated mutants often fail to colonize and rarely cause infections due to efficient opsonophagocytotic clearance by host cells (Magee, A.D. and Yother, J. 2001, Nelson, A.L., Roche, A.M., et al. 2007). Among S. pneumoniae species, serotype 3 is one of the most virulent (Briles, D.E., Crain, M.J., et al. 1992, Martens, P., Worm, S.W., et al. 2004, Weinberger, D.M., Harboe, Z.B., et al. 2010) and continues to be of concern despite its inclusion in the current conjugate vaccine against pneumococcal infections (Gruber, W.C., Scott, D.A., et al. 2012, Richter, S.S., Heilmann, K.P., et al. 2013, Wantuch, P.L. and Avci, F.Y. 2018, Wantuch, P.L. and Avci, F.Y. 2019). Armed with this knowledge, the benefits of utilizing an alternative therapy against type 3 pneumococcal infection becomes apparent. Indeed, we have begun investigations into utilizing Pn3Pase, described in this study, as a therapeutic agent against highly virulent type 3 infections (Middleton, D.R., Paschall, A.V., et al. 2018). Pn3Pase’s unique ability to hydrolyze the CPS of type 3 S. pneumoniae makes it an ideal therapeutic agent to reduce bacterial colonization and infection. Information from this current study will aid in further developing Pn3Pase as an alternative to conventional treatments such as antibiotics, establishing the biochemical characteristics necessary for optimum therapeutic potential. Additionally, recent work on a mucin-selective protease StcE (Malaker, S.A., Pedram, K., et al. 2019) suggests a possible role for the non-catalytic Pn3Pase mutants in purifying capsule. Type 3 is among the pneumococcal serotypes that has exhibited capsule shedding (Kietzman, C.C., Gao, G., et al. 2016). Non-active mutants of Pn3Pase may be useful in isolating shed capsule in vivo to assess capsule shedding in bacterial virulence as well as for serotyping in clinics.

In this current work we observed unique binding characteristics in our truncation mutants compared to non-catalytic full length proteins. TM3 mutant did not exhibit any significant binding to Pn3P substrate as well as having abolished activity. TM3 being non-catalytic was expected (Boraston, A.B., Bolam, D.N., et al. 2004), but we were surprised to see low-affinity binding to Pn3P substrate. However, it is noted that substrate binding for CBMs varies in a wide range with some having relatively weak interactions (Volkov, I.I.u., Lunina, N.A., et al. 2004) as could be the case in Pn3Pase CBM. TM2 recorded binding to Pn3P with approximately 10-fold lower affinity than full length proteins. An important role of the CBM is to target the substrate and bring it into closer proximity with the enzyme (Hervé, C., Rogowski, A., et al. 2010) which increases hydrolysis of the substrate (Shoseyov, O., Shani, Z., et al. 2006). TM2 lacks the putative CBM, which paired with the published work above may explain the lower binding affinity and no enzymatic activity. With no CBM, TM2 does not appear to bring Pn3P into close proximity with the hydrolase domain nor bind the substrate long enough for hydrolysis to occur. Several studies have suggested that removal of the CBM dramatically decreases enzymatic activity (Carrard, G. and Linder, M. 1999, Coutinho, J.B., Gilkes, N.R., et al. 1993, Shoseyov, O., Shani, Z., et al. 2006).

TM1 and E196A were the only mutants to display catalytic activity. We were unable to observe substrate binding with WT or TM1 due to degradation activity. Compared to WT, TM1 and E196A have significantly lower catalytic activities. In TM1 it is possible this could be due to structure and removal of the C terminal domains. InterPro analysis suggests full length Pn3Pase has two C-terminal domains that may possess carbohydrate-binding-like properties, the DUF1080 and a ConA-like/glucanase superfamily domain. Reviews have suggested proteins that possess hydrolytic activity, i.e. glycoside hydrolases, can have one or more CBMs (Shoseyov, O., Shani, Z., et al. 2006). If this is the case for Pn3Pase, TM1, being half the size of WT, could have lower catalytic activity due to removal of these other potential CBM-like domains. Additionally, the C terminal domains could be important for overall tertiary structure which could aid in activity. TM1 mutant does however demonstrate the minimum structure requirements for Pn3Pase activity, the glycoside hydrolase domain and putative CBM. All other C terminal domains do not appear to be essential for hydrolase activity. Our results also suggest the correct assignment of E196 as the acid/base catalytic residue and E306 as the nucleophile. This is apparent by E196A drastically decreased activity compared to WT and E306A complete loss of function. Prior work on GHs have shown that mutating these catalytic residues does not always lead to a complete ablation of activity (Armstrong, Z. and Davies, G.J. 2020). Additionally, it was previously suggested that the nucleophile alone may be sufficient for enzymatic hydrolysis (Henrissat, B., Callebaut, I., et al. 1996). This may explain why we observe activity when the acid/base residue is mutated, i.e., E196A, but not when the nucleophile is mutant (E306A). Further, members of clan GH-A have conserved glutamic acid residues functioning as the acid/base and nucleophile catalytic residues as appears to be the case with Pn3Pase (Henrissat, B., Callebaut, I., et al. 1996)

A Blast search using Pn3Pase sequence against the non-redundant protein sequence database of the NCBI shows that a small number of close homologs exist, all of which are essentially found in Paenibacillus sequences. Proteins with more distant similarity were also found (Table 2), but their distance to the only characterized GH169 member (Pn3Pase) is such that it is appropriate to place them into the non-classified CAZy section of the glycoside hydrolases until a sufficient taxonomical diversity is captured in family GH169. The reason behind the small size of the family is unclear; perhaps it simply means that degradation of Pn3P is not widespread and that the more distant relatives of Pn3Pase (Table 2) may act on a different substrate.

TABLE 2

Oligonucleotides for each protein used in this study

| | |
|---|---|
| Putative members of the family GH169 | Members of the non-classified CAZy section of the glycoside hydrolases |
| WP_079915027.1 | WP_037672359.1 |
| WP_028553222.1 | WP_145834095.1 |
| WP_126044750.1 | NHW38917.1 |
| WP_057315858.1 | WP_088980648.1 |
| WP_056617367.1 | AVV46075.1 |
| WP_068663733.1 | WP_159083366.1 |
| OMC67485.1 | WP_142165674.1 |
| WP-083655951.1 | SCL35799.1 |
| WP_113018908.1 | WP_113018907.1 |
| WP_113018909.1 | KMS75983.1 |
| WP_113675418.1 | WP_159029107.1 |
| | WP_141790907.1 |

Taken together, the work in this current study establishes the initial biochemical analysis of the glycoside hydrolase Pn3Pase which led to the establishment of GH family GH169. Despite our best efforts we have yet to obtain a crystal structure of this enzyme. Due to the large sequence divergence between Pn3Pase and other clan GH-A members we were also unable to build a homology model that had meaningful features. However, similarity with clan GH-A predicts Pn3Pase would have a β/α₈ barrel fold (TIM barrel), but the exact structural details that govern substrate recognition cannot be reliably predicted. Continuing work with Pn3Pase will involve a crystal structure to complement the work in this study and shed more light into the structure-function relationship of Pn3Pase with Pn3P. Additionally, further structural analysis will reveal if the correlations between activity and domains observed in this study are accurate and confirm the assignment of residues E196 and E306 as the acid/base and nucleophile. Further structural investigations will also aid in developing Pn3Pase as a therapeutic against type 3 pneumococcal infections.

### Material and Methods

### Production of Recombinant Pn3Pase and Mutants

WT recombinant Pn3Pase (DDBJ/ENA/GenBank accession number: MZNT01000000) was produced as previously described (Middleton, D.R., Zhang, X., et al. 2018). Briefly, the coding region of Pbac_3331 (Pn3Pase) was amplified from Paencibacillus sp. 32352 genomic DNA into pDONR221 using BP clonase reaction (Thermo Fisher Scientific). Primers for cloning WT and all mutants are listed in Table 1. After transformation into DH5α cells and DNA sequence confirmation, LR clonase reaction (Thermo Fisher Scientific) was performed to insert into pET-DEST42 destination vector for the expression of a carboxy-terminal His₆-tagged fusion protein in E. coli BL21(DE3) cells. C-terminal truncation mutants, TM1, TM2 and TM3 were produced by first amplifying DNA in PCR reaction containing 25 µl 2× master mix, 2.5 µl each of forward and reverse primers, 2.5 µl template Pbac. genomic DNA, and brought up to 50 ul with water. PCR reaction conditions were 98° C. 120 s, followed by 20 cycles of 98° C. for 15 s, 55° C. for 15 s and 72° C. for 6 mins. PCR products were visualized on 1% agarose gel and excised. DNA was extracted from gel using EZNA gel extraction kit (Omega Bio-tek). Using extracted DNA, mutants were then produced following the same BP and LR clonase reactions as WT above. Single and double site mutant proteins were produced using the Quik Change XL Site Directed Mutagenesis Kit (Agilent). PCR reactions contained 5 µl 10× buffer, 5 µl Pbac_3551 DNA in pDONR221 vector, 2.5 µl each of forward and reverse primers, 1 µl dNTPs, 3 uL. QuikSolution, 1 µl Pfu Ultra HF, and brought up to 50 ul with water. PCR reaction conditions were 95° C. for 60 s followed by 18 cycles of 95° C. for 50 s, 60° C. for 50 s and 68° C. for 9 mins. Following PCR Dpn1 reaction from kit was performed followed by transformation into XL10 competent cells and DNA sequence confirmation. LR clonase reaction was done same as above.

### Purification of Enzymes

BL21 cells transformed with pET-DEST42-Pn3Pase or mutant plasmid were grown in LB medium supplemented with 100 µg/mL ampicillin at 37° C. while cell density was monitored at absorbance 600 nm. Once OD 600 nm reached 1, cells were transferred to 20° C., protein expression was induced by adding Isopropyl β-D-1-thiogalactopyranoside to a final concentration of 1 mM and the cell culture was allowed to incubated for 24 hrs. Cells were harvested by centrifugation. Cells were resuspended in phosphate-buffered saline (PBS, pH 7.2) with 10 µg/mL DNase and lysed using EmulsiFlex-C5 homogenizer (Avestin). Lysate was cleared by centrifugation at 18,000 ×g for 45 mins at 4° C. and passed through a 0.45-um filter. Proteins were purified by Ni²⁺-NTA resin at 4° C. and eluted with 300 mM imidazole. Elution was run through FPLC Superdex 200 sizing column (GE LifeSciences) with PBS as running buffer. Protein concentration was determined using NanoDrop (Thermo Fisher Scientific) using extinction coefficients for each protein determined using ExPASy ProtParam tool (Gasteiger, E., Gattiker, A., et al. 2003). Purity was assessed by visualizing proteins on stain free tris-glycine gel (BioRad) using gel doc EZ imager (BioRad).

### Enzyme Activity Assays

Recombinant Pn3Pase and mutant hydrolysis activity was determined by measuring the increase in reducing ends using p-hydroxybenzoic acid hydrazide (PAHBAH) method (Blakeney, A.B. and Mutton, L.L. 1980). A reaction mixture (100 µl) containing 10 µg Pn3P and 30 nM, 60 nM or 120 nM enzyme in PBS was incubated at 37° C. for 30, 60 or 120 minutes then heated at 100° C. for 5 minutes to stop reaction. Time 0, used as a negative control, was the same reaction mixtures containing 30 nM, 60 nM or 120 nM of heat killed enzyme. 40 µl of reaction mixture was mixed with 120 µl of 1% (w/v) PAHBAH-HCI solution in duplicate, heated at 100° C. for 5 minutes. Absorbance at 410 nm was measured on Biotek synergy H1 microplate reader in a clear flat bottom 96-well microplate. Statistical analysis was determined using Student’s t test and compared all data points to their corresponding time zero data.

### Enzyme Kinetics

The Michaelis-Menten constant (K_{M}) and the maximum velocity (Vₘₐₓ) of WT Pn3Pase was measured using Pn3P as substrate (average molecular weight: 400,000 Da). The substrate was used at seven concentrations for WT (800, 400, 200, 100, 50, 25, and 0 nM) in PBS. WT Pn3Pase was added at 1 µg/ml (5.9 nM). Reactions were heated at 37° C. and aliquots were taken/stopped at 0, 4, 8, 12, 16, and 20 minutes (corresponding to approximately 10% of total hydrolysis yielding mostly tetrasaccharides) by heating reactions at 100° C. for 5 minutes. The amount of product formed was measured using PAHBAH-HCl assay as described above with tetrasaccharides, obtained from enzymatic degradation of Pn3P, used to generate a standard curve for data fitting. Initial velocity was determined using the amount of product formed in the linear region of absorbance. Initial velocities of each substrate concentration were curve fitted using non-linear regression with Michaelis-Menten model on GraphPad Prism to determine K_{M} and Vₘₐₓ. k_{cat} was likewise determined using GraphPad Prism as Vₘₐₓ/E_{T} with E_{T} set as total enzyme concentration 5.9 nM. The catalytic efficiency k_{cat}/Kₘ was determined using these values in min⁻¹M⁻¹. Tetrasaccharides used for standard curve were generated as previously described (Middleton, D.R., Zhang, X., et al. 2018). Standard deviation of data was determined through independent experimental duplicates.

### Binding Assay

Binding affinity of recombinant proteins to Pn3P substrate was determined using Biolayer Interferometry (BLI) (FortèBio OctetRED-384). All proteins and Pn3P were suspend in buffer (PBS, 0.5% bovine serum albumin [BSA], 0.05% Tween). Biotinylayed Pn3P substrate (20 µg/mL) was immobilized on streptavidin biosensor tips (FortèBio) for 150 s after an initial baseline in running buffer for 60 s. Baseline signal was measured again for 60 s before biosensor tips were immersed in wells containing protein (0 nm, 100 nM, 250 nM, or 500 nM) for 300 s. Dissociation was measured by returning biosensor tips to baseline for 300 s. Octet data analysis software was used to analyze data. Values of reference wells containing no protein (0 nm) were subtracted from data and affinity values were calculated using local and partial fit curve function with a 1:1 binding model. kₒₙ, k_{off} and K_{D} values were determined as the average of the three substrate concentration binding curves. Binding curves were graphed using GraphPad Prism.

### NMR for Stereochemical Analysis

¹H NMR analysis was performed on reaction products released from Pn3Pase hydrolysis of Pn3P to determine reaction mechanism. Pn3P was resuspended as 1 mg/mL (2.6 mM) in PBS and 10% D₂O and transferred to a 3 mm NMR tube. After collecting an initial spectrum at 37° C., the enzyme was added to the tube with a final concentration of 15 µg/mL, mixed, and data collection then resumed after 2 minutes. Data were collected continually and four transients were summed every 8 seconds. NMR spectra were acquired on an Agilent 600 MHz DD2 spectrometer equipped with a 3 mm cryoprobe, and used the standard presaturation pulse sequence to reduce the water signal. Data was processed with Mnova software (Mestrelab, Inc.)

### Generation of Biotinylated Pn3P

Biotinylated Pn3P was prepared using hydrazide biotin. 2 mg of 25 kDa Pn3P was dissolved in 500 µL of 0.1 M sodium borate buffer (pH 5.4). 12 mg of EZ-Link hydrazide-biotin (Thermo Fisher Scientific) was dissolved in 100 µL DMSO and added to Pn3P solution. 1.5 mg of 1-ethyl-3-(3-dimethylaminopropyl) carboiimide HCL (EDC) (Thermo Scientific) was added to the solution, vortexed and incubated before at 25° C. for 3 hours with agitation. Product was purified using Superdex 200 sizing column (GE LifeSciences).

### Citations for Example 1

Armstrong Z, Davies GJ. 2020. Structure and function of Bs164 β-mannosidase from Bacteroides salyersiae the founding member of glycoside hydrolase family GH164. J Biol Chem, 295:4316-4326.

Avery OT, Dubos R. 1930. THE SPECIFIC ACTION OF A BACTERIAL ENZYME ON PNEUMOCOCCI OF TYPE III. Science, 72:151-152.

Blakeney AB, Mutton LL. 1980. A simple colorimetric method for the determination of sugars in fruit and vegetables. J Sci Food Agric, 31:889-897.

Boraston AB, Bolam DN, Gilbert HJ, Davies GJ. 2004. Carbohydrate-binding modules: fine-tuning polysaccharide recognition. Biochem J, 382:769-781.

Briles DE, Crain MJ, Gray BM, Forman C, Yother J. 1992. Strong association between capsular type and virulence for mice among human isolates of Streptococcus pneumoniae. Infect Immun, 60:111-116.

Carrard G, Linder M. 1999. Widely different off rates of two closely related cellulose-binding domains from Trichoderma reesei. Eur J Biochem, 262:637-643.

Coutinho JB, Gilkes NR, Kilburn DG, Warren RAJ, Miller Jr RC. 1993. The nature of the cellulose-binding domain effects the activities of a bacterial endoglucanase on different forms of cellulose, 113 (2):211-217.

Davies G, Henrissat B. 1995. Structures and mechanisms of glycosyl hydrolases. Structure, 3:853-859.

Dubos R, Avery OT. 1931. DECOMPOSITION OF THE CAPSULAR POLYSACCHARIDE OF PNEUMOCOCCUS TYPE III BY A BACTERIAL ENZYME. J ExpMed, 54:51-71.

Gasteiger E, Gattiker A, Hoogland C, Ivanyi I, Appel RD, Bairoch A. 2003. ExPASy: The proteomics server for in-depth protein knowledge and analysis. Nucleic Acids Res, 31:3784-3788.

Gruber WC, Scott DA, Emini EA. 2012. Development and clinical evaluation of Prevnar 13, a 13-valent pneumocococcal CRM197 conjugate vaccine. Ann N Y Acad Sci, 1263:15-26.

Henrissat B, Bairoch A. 1996. Updating the sequence-based classification of glycosyl hydrolases. Biochem J, 316 ( Pt 2):695-696.

Henrissat B, Callebaut I, Fabrega S, Lehn P, Mornon JP, Davies G. 1996. Conserved catalytic machinery and the prediction of a common fold for several families of glycosyl hydrolases. Proc Natl Acad Sci U S A, 93:5674.

Hervé C, Rogowski A, Blake AW, Marcus SE, Gilbert HJ, Knox JP. 2010. Carbohydrate-binding modules promote the enzymatic deconstruction of intact plant cell walls by targeting and proximity effects. Proc Natl Acad Sci U S A, 107:15293-15298.

Kelley LA, Mezulis S, Yates CM, Wass MN, Sternberg MJ. 2015. The Phyre2 web portal for protein modeling, prediction and analysis. Nat Protoc, 10:845-858.

Kietzman CC, Gao G, Mann B, Myers L, Tuomanen EI. 2016. Dynamic capsule restructuring by the main pneumococcal autolysin LytA in response to the epithelium. Nat Commun, 7:10859.

Klontz EH, Trastoy B, Deredge D, Fields JK, Li C, Orwenyo J, Marina A, Beadenkopf R, Günther S, Flores J, et al. 2019. Molecular Basis of Broad Spectrum N-Glycan Specificity and Processing of Therapeutic IgG Monoclonal Antibodies by Endoglycosidase S2. ACS Cent Sci, 5:524-538.

Lombard V, Golaconda Ramulu H, Drula E, Coutinho PM, Henrissat B. 2014. The carbohydrate-active enzymes database (CAZy) in 2013. Nucleic Acids Res, 42:D490-495.

Magee AD, Yother J. 2001. Requirement for capsule in colonization by Streptococcus pneumoniae. Infect Immun, 69:3755-3761.

Malaker SA, Pedram K, Ferracane MJ, Bensing BA, Krishnan V, Pett C, Yu J, Woods EC, Kramer JR, Westerlind U, et al. 2019. The mucin-selective protease StcE enables molecular and functional analysis of human cancer-associated mucins. Proc Natl Acad Sci USA, 116:7278-7287.

Martens P, Worm SW, Lundgren B, Konradsen HB, Benfield T. 2004. Serotype-specific mortality from invasive Streptococcus pneumoniae disease revisited. BMC Infect Dis, 4:21.

Middleton DR, Lorenz W, Avci FY. 2017. Complete Genome Sequence of the Bacterium. Genome Announc, 5.

Middleton DR, Paschall AV, Duke JA, Avci FY. 2018. Enzymatic Hydrolysis of Pneumococcal Capsular Polysaccharide Renders the Bacterium Vulnerable to Host Defense. Infect Immun.

Middleton DR, Zhang X, Wantuch PL, Ozdilek A, Liu X, LoPilato R, Gangasani N, Bridger R, Wells L, Linhardt RJ, et al. 2018. Identification and characterization of the Streptococcus pneumoniae type 3 capsule-specific glycoside hydrolase of Paenibacillus species 32352. Glycobiology, 28:90-99.

Mitchell AL, Attwood TK, Babbitt PC, Blum M, Bork P, Bridge A, Brown SD, Chang HY, El-Gebali S, Fraser MI, et al. 2019. InterPro in 2019: improving coverage, classification and access to protein sequence annotations. Nucleic Acids Res, 47:D351-D360.

Moxon ER, Kroll JS. 1990. The role of bacterial polysaccharide capsules as virulence factors. Curr Top Microbiol Immunol, 150:65-85.

Nelson AL, Roche AM, Gould JM, Chim K, Ratner AJ, Weiser JN. 2007. Capsule enhances pneumococcal colonization by limiting mucus-mediated clearance. Infect Immun, 75:83-90.

Richter SS, Heilmann KP, Dohrn CL, Riahi F, Diekema DJ, Doern GV. 2013. Pneumococcal serotypes before and after introduction of conjugate vaccines, United States, 1999-2011(1.). Emerg Infect Dis, 19:1074-1083.

Shoseyov O, Shani Z, Levy I. 2006. Carbohydrate binding modules: biochemical properties and novel applications. Microbiol MolBiol Rev, 70:283-295.

Sickles GM, Shaw M. 1934. A Systematic Study of Microörganisms Which Decompose the Specific Carbohydrates of the Pneumococcus. J Bacteriol, 28:415-431.

Volkov IIu, Lunina NA, Velikodvorskaia GA. 2004. Prospects for practical application of substrate-binding modules of glycosyl hydrolases. PriklBiokhim Mikrobiol, 40:499-504.

Wantuch PL, Avci FY. 2018. Current status and future directions of invasive pneumococcal diseases and prophylactic approaches to control them. Hum Vaccin Immunother, 14:2303-2309.

Wantuch PL, Avci FY. 2019. Invasive pneumococcal disease in relation to vaccine type serotypes. Hum Vaccin Immunother: 1-2.

Weinberger DM, Harboe ZB, Sanders EA, Ndiritu M, Klugman KP, Rückinger S, Dagan R, Adegbola R, Cutts F, Johnson HL, et al. 2010. Association of serotype with risk of death due to pneumococcal pneumonia: a meta-analysis. Clin Infect Dis, 51:692-699.

The complete disclosure of all patents, patent applications, and publications, and electronically available material (including, for instance, nucleotide sequence submissions in, e.g., GenBank and RefSeq, and amino acid sequence submissions in, e.g., SwissProt, PIR, PRF, PDB, and translations from annotated coding regions in GenBank and RefSeq) cited herein are incorporated by reference in their entirety. Supplementary materials referenced in publications (such as supplementary tables, supplementary figures, supplementary materials and methods, and/or supplementary experimental data) are likewise incorporated by reference in their entirety. In the event that any inconsistency exists between the disclosure of the present application and the disclosure(s) of any document incorporated herein by reference, the disclosure of the present application shall govern. The foregoing detailed description and examples have been given for clarity of understanding only. No unnecessary limitations are to be understood therefrom. The disclosure is not limited to the exact details shown and described, for variations obvious to one skilled in the art will be included within the disclosure defined by the claims.

Unless otherwise indicated, all numbers expressing quantities of components, molecular weights, and so forth used in the specification and claims are to be understood as being modified in all instances by the term “about.” Accordingly, unless otherwise indicated to the contrary, the numerical parameters set forth in the specification and claims are approximations that may vary depending upon the desired properties sought to be obtained by the present disclosure. At the very least, and not as an attempt to limit the doctrine of equivalents to the scope of the claims, each numerical parameter should at least be construed in light of the number of reported significant digits and by applying ordinary rounding techniques.

Notwithstanding that the numerical ranges and parameters setting forth the broad scope of the disclosure are approximations, the numerical values set forth in the specific examples are reported as precisely as possible. All numerical values, however, inherently contain a range necessarily resulting from the standard deviation found in their respective testing measurements.

All headings are for the convenience of the reader and should not be used to limit the meaning of the text that follows the heading, unless so specified.

1. A non-natural Pn3Pase protein comprising an amino acid sequence of at least 80% identity with amino acids 41-765 of SEQ ID NO:2.

2. The protein of claim 1 further comprising at least one heterologous amino acid at the amino-terminal end, the carboxy-terminal end, or both amino- and carboxy-terminal ends.

3. (canceled)

4. The protein of claim 2 wherein the heterologous amino acids comprise a tag.

5-7. (canceled)

8. A genetically modified cell comprising an exogenous polynucleotide comprising a coding region, wherein the coding region comprises a nucleotide sequence encoding the protein of claim 1.

9. The genetically modified cell of claim 8 wherein the cell is a eukaryotic cell.

10. The genetically modified cell of claim 9 wherein the cell is a mammalian cell, a yeast cell, or an insect cell.

11. The genetically modified cell of claim 8 wherein the cell is a prokaryotic cell.

12. The genetically modified cell of claim 11 wherein the cell is E. coli.

13-16. (canceled)

17. A method comprising:
incubating the genetically modified cell of claim 8 under conditions suitable for expression of the protein.

18. The method of claim 17 further comprising isolating the protein.

19. The method of claim 17 further comprising purifying the protein.

20. The method of claim 17 wherein the cell is a eukaryotic cell.

21. The method of claim 20 wherein the cell is a mammalian cell, a yeast cell, or an insect cell.

22. The method of claim 17 wherein the cell is a prokaryotic cell.

23. The method of claim 22 wherein the prokaryotic cell is E. coli.

24. (canceled)

25. A method comprising:
contacting a Streptococcus pneumoniae comprising a type III capsular polysaccharide with the non-natural Pn3Pase protein of claim 1, wherein the contacting is under conditions suitable for enzymatic hydrolysis of type III capsular polysaccharide, wherein the amount of type III capsular polysaccharide on the surface of the S. pneumoniae is reduced compared to the S. pneumoniae that is not contacted with the non-natural Pn3Pase protein.

26. (canceled)

27. The method of claim 25 wherein the S. pneumoniae is present in conditions suitable for replication of the S. pneumoniae.

28. The method of claim 1 wherein the contacting comprises exposing the type III capsular polysaccharide or the S. pneumoniae to a genetically modified cell that expresses the non-natural Pn3Pase protein.

29. The method of claim 25 wherein the S. pneumoniae has increased susceptibility to phagocytosis by macrophages, increased complement-mediated killing by neutrophils, or a combination thereof, compared to the S. pneumoniae that is not contacted with the non-natural Pn3Pase protein.

30. A method for treating an infection in a subject, the method comprising:
administering an effective amount of the non-natural Pn3Pase protein of claim 1 to a subject having or at risk of having an infection caused by a serotype 3 S. pneumoniae.

31-38. (canceled)


SEQUENCE LISTING (19 sequences)

SEQ ID NO: 1 - DNA, 4638 bp, Paenibacillus sp 32352
atggctattggaacatcatataggctgcgtcaggcattgaaccgatcctgtatcgcgcttgcggccagtgccttgctggcatcggctgcagcaagcttcaccgcaggaacgactcatgcggcacccgtgaatctggaagcggattatgccttaagtgaagggcagcttgtccgcacggagcaattcaacaatacgaattacacgccgcttccggttcatgtcgtcgatgaactcaaagggatccggaccaaggtcgtaagagattttgttaaaattaattggtactacaacaaggatgaggcgaacagcgattatttggcttattccatcgacgacccgcgtaacgcgacgaacccggatttacagcatggccgcaaggaaacgtatgatttcatgtcccagtttaccgatggcattctcctatctctcgcttattcctatggaggtgacgcgaatccgcagaaaaaccgcctcctagccggcgagaccgaaatgaactggcctgaattcgacaaggcgatgaagaaaatcatttatacgttgaaggagaaaaatccgaagatcgaatatatcgaggtgggcaacgagccgaacctggagcctgcctattatggacatgtgaaggatgatattcccggctacatgcgcatgtatcagggcatgtcggaagcggtaaaatgggtaaacgcacagggcttggccgggcagccgctgaaagtaggcggaccggtattatccggctacaatttcgagaagcagaagcagttcgtcgatcttgcctacgccgcaagctatcaggtcgattttgtgtcatggcaccgataccaggaggacgtgcggatgaacgaaacgcaggaaatcgaaatgaaaagctatctgcacaagttttatccgaatgctataaccattgttagcgaatacggctggaaggggggcgggggcttaagtgatccgacgaacaacgtcggactggcgaagcaagcggcgtttatgacggactcggcatcctattatgcccgtggagggacggatattccgatgaactgggtagcggtccatacactgaatgcgtatttcaagaaccagttcgatgtcgactatgctttaagcaatggggatacgacgaattggcagacatttatgaatcctaatccaagggctttacaatatttgaacctgcgcgggtggagggagtcggcatcgacctccaagccgatgaagatcaaggaaattcgtttttttgacagcagtaacaacccgattccaatcccgaatccggcaaacgacccgaatattgcggcggttacggacggtgacgatgcgacgcagtttatccaagcagactactggacctggctcaagtttgatctggggccatcggctcccgggattgccagggttgatatcaagtgggggaatcccgatatcaacaccttccagctcattggcactagtgataaattgaaatattacgaggcgctgggccgcactttctacaccccgtattttaacacaatgcggatgttcgcccggctcggggacacccgggtgaaagcaaccggcggcagcagtgacatctatgggacacggatgctggcgacgaagaacagcgattccaaagcgacgatgatggtgtggaacaaacagggagacggggcggcctcatccaatgtcagcatcgatgtgaagcatctgccgccaggctttgaagggaagagtgtacggatcaaaaagtatttggtcgacgagacccatagcaattatgcctacaacaaaatcgacgagctccagttgttcgacgaaaccatagcgaacacggcggacaatctgacgattaacgctacgttgaatagaaatgcggtaatgctgatagagctggaggcggttgatcctagcatccaaaatatcgtgtctgccggcaaggaagttacctctacgacaggaatgacgaatatgccggcgctggtggacggcaatagtctgactgctgctgtcgcagccgatagcagctatccgcaaactatacaaatggatctcggcagaccctatgagttatcagggatgcaaatcgattggacgaacagcgcatcgagagatttcagctacgcaatagcgacgagcttggatggcacgtcgtaccatacggtagcggatcaaaccgacccggcttccgcagtcccaagagggaacagtttggactggttccaaaccaaggcgcggtatgtgaagctgacggtgacgcgctccaccttcgatggcccgctatcgattaacgatgtaaaggtttttgccgaagcgctgtacaaaaacgggttcgacaccaccgaggataagaatgcgatggcggcatggacgacggttggctttggatcgaaatccaccccatggaccaccgtgacggacagtgtttaccataacacattcgtgcaaccgatggacctattcagcacgacacctagcttcgccatttttggtgacagtcttaaggattatggagtggaagcgcgggtcagaactacggacagtggaaacgtgcaaatggggcttattgcccgagcgaccgattacaataacttgtattacttcaagctgtaccgaagcagctccgcgcatcaggcagtgctggagaagcgcgtcaatggaacgacaaccgtgcttaagtcggtggatttggctgatccgattcagtcgaaccgctggtataggctgaatttggaagctgtcggaacaacgctgcgagcttctattgacggtgttcaggttatcgagcttacggatgccagccgggcttccggtaaattcgggctgcgaagccatgaatccatcgccggcttcgacgatgtcaaagtgtatccgattgttccgctgcttggcgatattaaggtgaacagcgtatctattgagggcttccatccgcatttgaacaactacactgtacttgtcccatcgacgagctctgcagtaaccgtgacggcaaccgtctacggtacggccaataccgtggcgtccccggcctccgggcaggtgaccttcgacgcggttggccaagagaggtcgtacacagtagctgcgctgtcgactgaaggcaatggggcgaattattacaagataaacttgcggacggcaagcgccgatacatcgcttagcaacctgaggctgtcggtcattacggacgctctgacagccccgggccaacaattacccgaatcgatcgttgctctggttccgggtcaaacggaatacacgattcacgttccttcccgtacggcttatgtcaaggtggtagaggcgatcccgacggtaagcaatgtggcttcagttcaaatcactaacgcaactttggtgaacggctcgggtaccgcaacggtaaaagtaacctccgaagccggaacgacggcggaatatatgctgaatatccgggcgaacccggaggcggctgccgggacggtgctgtacgaagaaaattttgagagcggagcttacaatcaagacgcagcgaccggttggaataacggtgccgtaccgaatggtgcagcttcacatctacgcgttgcggatgaggatcagggcaaggtgctggagaagtatacgacgaccagcatggcctttacggtcggtcaaagcggttggacagattacgatgtaagggccagagttaaagcgcaggccagcccgtcactgccaggcattattgcgcgcgcatccgacgatgccaaaaacttttatatgctgcgcattcataacggcacgaatggactgcctggcggaagcacggggtatgtcattcttggccgtgtcgtgaacgggacgctccgcgagctggacagcaaaaaaatcccatacccgtatgtggtcggtaactggtatcagcttcgtcttgtcgtggatagtaatcggatcaaaggctatgtggacgatagcctgatctatgacgaagtagataacggatcgttattcccgacgaatcctcctgcactgatgcaaggcaaggcggggatccgggtggcaaaccaggcagcccgcatcgatgattttcaggtatcccatcttgctgccctacccgcagattcggtgaagccggtgataaccctgaacgggccggcagtggttgaagtattggctggcagcacgtatacggatgcaggagcgacggcggcggatgatgtggacggcgatctgacaagccgcattcaagtgagcggcagtgtggacacgtcgacgccaggaacgtacacgctggtctataccgtaatggacaacgcacataatgtagcggacccggttgtacgcactgtaacggtaaccgctgcgggggataagccgtttactgtcattgcaaatggcgggctgaaccggacgaacggtttgagtgctaaggtcgacgttacccgcacacccggctcggtagatcatgcgggtagggaagtcgtttactttcagcttcttaagggcagcactcccgtatcccatgttgcgttggaaagcgagcttacgaatggcggcagcctgacaggccatttcgatgtgccggatgctgaagagggcacttatacggttcatgtgtttatcatcgatatgctcgactctatagagggatccttgccgattccgttatcgaataaggtgatcgtggagtag

SEQ ID NO: 2 - protein, 1545 aa, Paenibacillus sp 32352
MAIGTSYRLRQALNRSCIALAASALLASAAASFTAGTTHAAPVNLEADYALSEGQLVRTEQFNNTNYTPLPVHVVDELKGIRTKVVRDFVKINWYYNKDEANSDYLAYSIDDPRNATNPDLQHGRKETYDFMSQFTDGILLSLAYSYGGDANPQKNRLLAGETEMNWPEFDKAMKKIIYTLKEKNPKIEYIEVGNEPNLEPAYYGHVKDDIPGYMRMYQGMSEAVKWVNAQGLAGQPLKVGGPVLSGYNFEKQKQFVDLAYAASYQVDFVSWHRYQEDVRMNETQEIEMKSYLHKFYPNAITIVSEYGWKGGGGLSDPTNNVGLAKQAAFMTDSASYYARGGTDIPMNWVAVHTLNAYFKNQFDVDYALSNGDTTNWQTFMNPNPRALQYLNLRGWRESASTSKPMKIKEIRFFDSSNNPIPIPNPANDPNIAAVTDGDDATQFIQADYWTWLKFDLGPSAPGIARVDIKWGNPDINTFQLIGTSDKLKYYEALGRTFYTPYFNTMRMFARLGDTRVKATGGSSDIYGTRMLATKNSDSKATMMVWNKQGDGAASSNVSIDVKHLPPGFEGKSVRIKKYLVDETHSNYAYNKIDELQLFDETIANTADNLTINATLNRNAVMLIELEAVDPSIQNIVSAGKEVTSTTGMTNMPALVDGNSLTAAVAADSSYPQTIQMDLGRPYELSGMQIDWTNSASRDFSYAIATSLDGTSYHTVADQTDPASAVPRGNSLDWFQTKARYVKLTVTRSTFDGPLSINDVKVFAEALYKNGFDTTEDKNAMAAWTTVGFGSKSTPWTTVTDSVYHNTFVQPMDLFSTTPSFAIFGDSLKDYGVEARVRTTDSGNVQMGLIARATDYNNLYYFKLYRSSSAHQAVLEKRVNGTTTVLKSVDLADPIQSNRWYRLNLEAVGTTLRASIDGVQVIELTDASRASGKFGLRSHESIAGFDDVKVYPIVPLLGDIKVNSVSIEGFHPHLNNYTVLVPSTSSAVTVTATVYGTANTVASPASGQVTFDAVGQERSYTVAALSTEGNGANYYKINLRTASADTSLSNLRLSVITDALTAPGQQLPESIVALVPGQTEYTIHVPSRTAYVKVVEAIPTVSNVASVQITNATLVNGSGTATVKVTSEAGTTAEYMLNIRANPEAAAGTVLYEENFESGAYNQDAATGWNNGAVPNGAASHLRVADEDQGKVLEKYTTTSMAFTVGQSGWTDYDVRARVKAQASPSLPGIIARASDDAKNFYMLRIHNGTNGLPGGSTGYVILGRVVNGTLRELDSKKIPYPYVVGNWYQLRLVVDSNRIKGYVDDSLIYDEVDNGSLFPTNPPALMQGKAGIRVANQAARIDDFQVSHLAALPADSVKPVITLNGPAVVEVLAGSTYTDAGATAADDVDGDLTSRIQVSGSVDTSTPGTYTLVYTVMDNAHNVADPVVRTVTVTAAGDKPFTVIANGGLNRTNGLSAKVDVTRTPGSVDHAGREVVYFQLLKGSTPVSHVALESELTNGGSLTGHFDVPDAEEGTYTVHVFIIDMLDSIEGSLPIPLSNKVIVE

SEQ ID NO: 3 - skipped

SEQ ID NO: 4 - protein, 775 aa, PAENIBACILLUS SP. YN15
MNNMISRKGWKQAKRPFSALTAGMLAVSMALGLAPAFPETAQAEVSPTAQVLTADYGTSAGPLIRTEQFNNTNYAPLPVHVVDELKGIKTKIVRDFVKINWYYNKDPNNSDLLAYSIETPENLETNPDLQHGRKETYDFMGQFADSILISLAYSYGGDTNPGKNRLLSGETTMNWDEYDKAMRVIIKTLKEKNPKLEYIEVGNEPNLEPAYFGHVKDDIPGYMRMYEGMSKAVVWVNQQLGLNDTFGSNGVRLKVGGPVLSGYNFAKQKEFVDIAYQNHYQVDFVSWHRYRQEITENETQAIQMRNYLSDKYPDATLIVSEYGWKGGGGLTDSTNNVALAKQAAFMTDSAYFYERGGVDIPMNWVAVHTLNAYFKNQFDVDYALSNGTVGWQEFNSNYTDPVRYMNLRGWRESATTSGRQVKIQEIEFYDPAGNKIAVPNTAGDPLIAVATDGDESTQFLQSDYWTWLRFDLGAEYQISKIRIKWGDSQVYKFQVVGTSDKLKYHELLGKTFFTPYFNTMRMLSRLGDEKVAVTGSDTGNTGVRLLATKNSDSKATMMVWNHQLDGKASKEVSVQVKNLPASFQGKSIRYKKYLVDATHSNYAYNKNDSLEVVGQGTMNITGTEIFNETLTPNAVMLIELEAVDSSINNIVSAGKTATGDLQNLAALVDGDERTSAAAANNSYPQSVVIDLERNFTVSGAEIKWSNAELQSYHYSISTSTDGGSYSTVVDSTYGAWSKGSSLHWFPATARYVKLTVTGSYTGQPLSIDDISIFAD

SEQ ID NO: 5 - protein, 759 aa, PAENIBACILLUS ORYZISOLI
MGLLKKHCAALTAAIMLTSAAVSLAPQAAQAASMNMEADYAVSEGPLVRTEQFNNTNYTPLPVHVVDELKGIKTKVVRDFVKINWYYNKDSANSDYLAYSIDDARNATNPDLLSARKETYDFMSQFSDSLLISLAYSYGGDANPEKNRLIAGETTMNWPEFDKAMKKIIFTLKEKNPELEYIEVGNEPNLEPAFYGHTKEDIPGYMRMYQGMSEAVKWVNTQGLAGQSVKVGGPVLSGYNFDKQKQFVDIAYANGYQVDFVSWHRYQEDVRMNETQEIEMKGYLHRFYPNATTIVSEYGWKGGGDLSDATNNVGLAKQAAFMTDSAYFYARGGTDIPMNWVAVHTLNAYFKNQFDVDYALSTGDTTNWQSFTNSDPRSLQYLNLRGWRESASTSKPMKIKEIRFFDSNNNPIPIPNVLNDPSIAAVTDNDDATQFTQSDYWTWLKFDLGTNAPAVARVDIKWGNTDINTFQLIGTSDKLKYYEVLGHTFFTPYFNTMRMFAQLGDTRVKTSGGSMEIYGTRMLATKNSDTKATMMVWNKQGDGTASANVDISVKNLPAGFQGKSVRYKKLLVDESHSNHAYNKRDDLQMVDEGIVNLTDTIVLSQTLEKNAVMLIELEAVDPSIKNIVSAGKAVTLSPGMTGGANLVDGYDTTAAVAATNTYPQTVTVDLGKSYQLAGMGIDWTNSASRGYTYTIESSLDGTHFTLASDMTFSSNPTYVPPLGNSLAWFTGKARYVKLTVTGSTYDGPLSINEVKVFAD

SEQ ID NO: 6 - protein, 759 aa, PAENIBACILLUS SP. SOIL766
MGLLKKHYAALTAALMLTSAAVSFAPQSVHAASVNMEADYAVSEGPLVRTEQFNNTNYTPLPVHVVDELKGIKTKVVRDFVKINWYYNKDSANSDYLAYSIDDARNATNPDLLSARKETYDFMSQFSDSLLISLAYSYGGDANPEKNRLIAGETTMNWIEFDKAMKKIIYTLKEKNPELEYIEVGNEPNLEPAFYGHTKEDIPGYMRMYQGMSEAVKWVNTQGLTGQSVKVGGPVLSGYNFDKQKQFVDIAYANGYQVDFVSWHRYQEDVRMNETQEIQMKTYLHQFYPNATTIVSEYGWKGGGGLSDATNNVGLAKQAAFMTDSAYFYARGGTDIPMNWVAVHTLNAYFKNQFDVDYALSNGDTTNWQSFTNSDPRSLQYLNLRGWRESASTSKPMKIKEIRFFDSSNNPIQIPNVINDPSIAAVTDNDDATQFTQSDYWTWLKFDLGANAPAIARVDIKWGNTDINTFQLIGTSDKLKYYEVLGHTFFTPYFNTMRMLAQLGDMRVKATGGSTDIYGIRMLATKNSDTKATMMVWNKQGDGAASANVDISVKNLPAGFQGKSVRYKKLLVDESHSNHAYNKIDDLQMVDEGIVNLTDTIVLSQTLEKNAVMLIELEAVDPSIKNIVSAGKTVTLSPGMTGGANLVDGHDTTAAVSATNTYPQTVTVDLGKSYQLAGMGIDWTNSASRGYTFKIETSLDGVNFTLASDMTFSSNPAYVPPLGNSLAWFTGKARYVKLTVTGSTYDGPLSINEVKLFAD

SEQ ID NO: 7 - skipped

SEQ ID NO: 8 - protein, 759 aa, PAENIBACILLUS SP. SOIL750
MGLFKKHCAALTAALMLSSVAISFAPQSVHAASVNMEADYAVSEGPLVRTEQFNNTNYTPLPVHVVDELKGIKTKVVRDFVKINWYYNKDSANSDYLAYSIDDARNATNPDLLSARKETYDFMSQFSDSLLISLAYSYGGDANPEKNRLIAGETTMNWPEFDKAMKKIIYTLKEKNPELEYIEVGNEPNLEPAFYGHTKDDIPGYMRMYQGMSEAVKWVNTQGLTGHSVKVGGPVLSGYNFDKQKQFVDIAYANGYQVDFVSWHRYQEDVRMNETQEIQMKGYLHQFYPNATTIVSEYGWKGGGGLSDATNNVGLAKQAAFMTDSAYFYARGGTDIPMNWVAVHTLNAYFKNQFDVDYALSNGDTMNWQSFTNSDPRSLQYLNLRGWRESASTSKPMKIKEIRFFDSSNNLIPIPNALNDPSIAAVTDNDDATQFTQSDYWTWLKFDLGSNAPAIARVDIKWGNTDINTFQLIGTSDKLKYYEVLGHTFFTPYFNTMRMFAQLGDTRVKASGGSTDIYGTRMLATKNNDTKATMMVWNKQGDGTASANVDISVKNLPAGFQGKSVRYKKMLVDESHSNHAYNKIDDLQMVDEGIVNLTDTIVLSQTLEKNAVMLIELEAVDPSIKNIVSAGKTVTLSPGMTGGANLVDGYETTAAVAATNTYPQTVTVDLGKSYQLAGMGIDWTSSASRGYTYKIETSLDGVNFTLASDMTFSSNPAYVPPLGNSLAWFTGKARYVKLTVTGSTYDGPLSINEVKVFAD

SEQ ID NO: 9 - protein, 759 aa, PAENIBACILLUS SP. URHA0014
MGLLRKHCAALTAALMLTSVAVSFAPQSVHAASVNMEADYAVSEGPLVRTEQFNNTNYTPLPVHVVDELKGIKTKVVRDFVKINWYYNKDSTNSDYLAYSIDDARNANNPDLLSARKETYDFMSQFSDSLLISLAYSYGGDANPEKNRLIAGETTMNWPEFDKAMKKIIYTLKEKNPKLEYIEVGNEPNLEPAFYGHTKEDIPGYMRMYQGMSEAVKWVNTQGLTGQSVKVGGPVLSGYNFDKQKQFVDIAYANGYQVDFVSWHRYQEDVRMNETQEIEMKGYLHRFYPNATTIVSEYGWKGGGGLSDATNNVGLAKQAAFMTDSAYFYARGGTDIPMNWVAVHTLNAYFKNQFDVDYALSNGDTTNWQSFTNSDPRSLQYLNLRGWRESASTSKPMKIKEIRFFDSSNNPIPIPNVLNDPSIAAVTDNDDATQFTQSDYWTWLKFDLGSNAPAIARVDIKWGNTDINTFQLIGTSDKLKYYEVLGHTFFTPYFNTMRMFAQLGDTRVKTSGGSTDIYGTRMLATKNSDTKATMMVWNKQGDGTASANVDISVKNLPAGFQGKSVRYKKMLVDESHSNHAYNKVDDLQMVDEGIVNLTGTIVLSQTLDKNAVMLIELEAVDPSIKNIVSAGKTVTLSQGMTGGANLVDGYDTTAAMAANNTYPQTVTVDLSKPYQLAGMGIDWTYSASRGYTYKIETSLDGINFTLASDKTFSSNPAYVPPLGNSLAWFTGKARYVKLTVTGSTYDGPLSINEVKVFAD

SEQ ID NO: 10 - DNA, 69 bp, Artificial
  oligonucleotide
ggggacaagtttgtacaaaaaagcaggcttcgaaggagatagaaccatggcacccgtgaatctggaagc

SEQ ID NO: 11 - DNA, 56 bp, Artificial
  oligonucleotide
ggggaccactttgtacaagaaagctgggtgctccacgatcaccttattcgataacg

SEQ ID NO: 12 - DNA, 52 bp, Artificial
  oligonucleotide
ggggaccactttgtacaagaaagctgggtgttcggcaaaaacctttacatcg

SEQ ID NO: 13 - DNA, 48 bp, Artificial
  oligonucleotide
ggggaccactttgtacaagaaagctgggtgggtgtagaaagtgcggcc

SEQ ID NO: 14 - DNA, 17 bp, Artificial
  oligonucleotide
gggcaacgcgccgaacc

SEQ ID NO: 15 - DNA, 17 bp, Artificial
  oligonucleotide
ggttcggcgcgttgccc

SEQ ID NO: 16 - DNA, 22 bp, Artificial
  oligonucleotide
tgttagcgcatacggctggaag

SEQ ID NO: 17 - DNA, 22 bp, Artificial
  oligonucleotide
cttccagccgtatgcgctaaca

SEQ ID NO: 18 - DNA, 67 bp, Artificial
  oligonucleotide
ggggacaagtttgtacaaaaaagcaggcttcgaaggagatagaaccatgaatatcgtgtctgccggc

SEQ ID NO: 19 - DNA, 52 bp, Artificial; identical to SEQ ID NO: 12
  oligonucleotide